(19) United States
(12) Patent Application Publication
(10) Pub. No.: US 2025/0154177 A1
(43) Pub. Date: May 15, 2025

(54) PYRROLOBENZODIAZEPINE INTERMEDIATES AND USES THEREOF

(71) Applicant: Sigma-Aldrich Co. LLC, St. Louis, MO (US)

(72) Inventors: James Walker, Verona, WI (US); Emilia Groso, Madison, WI (US); Austin Wright, Madison, WI (US); David Goeddel, Middleton, WI (US); Sheshagiri P T, Bangalore (IN); Sachin Tanaji Aiwale, Bangalore (IN); Ravindra Vikram Singh, Bangalore (IN); Ross Bemowski, Madison, WI (US); Louis Redfern, Evanston, IL (US); Logan Berg, Madison, WI (US)

(21) Appl. No.: 18/838,939

(22) PCT Filed: Mar. 10, 2023
(86) PCT No.: PCT/US2023/064151

§ 371 Date: Aug. 15, 2024

Related U.S. Application Data
(60) Provisional application No. 63/269,205, filed on Mar. 11, 2022.

(51) Int. Cl. C07F 7/18; C07D 487/04

(52) U.S. Cl. CPC C07F 7/1804; C07D 487/04

(57) ABSTRACT

Pyrrolobenzodiazepine (PBD) intermediates, including useful in the preparation of advanced intermediates. PBDs, and PBD dimers. Such PBD intermediates provide several advantages, including simplified synthesis of desired PBD products, greater substrate compatibility, and safer reaction conditions.

### CROSS-REFERENCE TO RELATED APPLICATIONS

The application claims the benefit of priority of U.S. Provisional Application No. 63/269,205, filed Mar. 11, 2022, the entire contents of which are incorporated herein by reference.

### BACKGROUND

Pyrrolobenzenodiazepines (PBDs), discovered in 1963, are a class of naturally occurring compounds having antibiotic and antitumor properties. Originally derived from streptomyces, PBDs are potent DNA damaging agents that are significantly more potent than current systemic chemotherapeutic drugs. While toxicity concerns limited initial applications, PBDs have been shown to be effective as payloads in antibody-drug conjugates (ADCs). There is currently one commercial PBD-containing ADC, with many other currently in clinical trials.

Currently, no commercially available intermediates exist for the synthesis of PBD payloads. Scientists must develop each synthesis de novo, then further scale up and develop GMP methods for manufacturing PBDs. A need exists for new intermediates useful in the synthesis of PBDs.

### SUMMARY

Provided are compounds of formula I useful in the synthesis of pyrrolobenzodiazepines

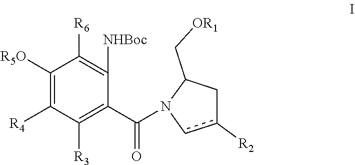

wherein the broken line represents a single or double bond.

R₁ is a protecting group selected from tert-butyldimethylsilyl ether (TBS) and acetate (Ac).

R₂ is selected from —H, ═O, —OTf, —OH, C₁-C₅ alkyl, C₃-C₆ cycloalkyl, C₁-C₅ alkenyl, C₅-C₁₀ aryl, C₅-C₁₀ heteroaryl, and a linker. Optionally, R₂ may be substituted with one or more substituents selected from halo, nitro, cyano, ether, carboxy, ester, C₁-C₇ alkyl, C₁-C₇ alkenyl, C₃-C₇ heterocyclyl, bis-oxy-C₁-C₃ alkylene and a linker.

R₃ is selected from —H, —OH, —OR, —SH, —NH₂, —NO₂, halo, wherein R is selected from C₁-C₄ alkyl, C₃-C₁₀ heterocyclyl and C₅-C₁₀ aryl. R may optionally be substituted with a C₁-C₄ alkyl or phenyl.

R₄ is selected from —H, —R, —OH, —OR, —SH, —SR, —NH₂, —NHR, —NHRR′, nitro and halo, wherein R and R′ are independently selected from C₁-C₄ alkyl, C₃-C₁₀ heterocyclyl and C₅-C₁₀ aryl. R and R′ may optionally have a substituent selected from C₁-C₄ alkyl and phenyl.

R₅ is a protecting group selected from tert-butyldiphenylsilyl ether (TBDPS) and triisopropylsilyl ether (TIPS).

R₆ is selected from —H, —OH, —OR, —SH, —NH₂, —NO₂, halo, wherein R is selected from C₁-C₄ alkyl, C₃-C₁₀ heterocyclyl and C₅-C₁₀ aryl, and R may optionally have a substituent selected from C₁-C₄ alkyl, phenyl.

In some embodiments, R₃ and R₆ are each —H.

In some embodiments, R₂ is selected from —H, ═O, —OTf, —OH, methyl, cyclopropyl, ═CH₂, ethylenyl, propenyl, phenyl, 4-fluorophenyl, indole and a linker. R₂ may be optionally substituted with one or more substituents selected from halo, nitro, cyano, ether, carboxy, ester, C₁-C₇ alkyl, C₁-C₇ alkenyl, C₃-C₇ heterocyclyl, bis-oxy-C₁-C₃ alkylene and a linker.

In some embodiments, R₄ is selected from —H, —OCH₃, —OCH₂Ph, —N(CH₃)₂, morpholino, piperidinyl, and —N—Me-piperazinyl.

In some embodiments R₂ is ═O and in some embodiments, R₂ is-OTf.

In some embodiments, R₄ is OCH₃.

Also provided are methods for making an advanced PBD intermediate, the method including the steps of providing a compound of formula I wherein R₂ is a ketone, performing a C—C bond forming reaction at the ketone, and optionally, deprotecting the amine.

In some embodiments, the method includes the step of converting the ketone to a triflate, then performing a C—C bond forming reaction on the triflate, and optionally, deprotecting the amine.

In some embodiments, the C—C bond forming reaction is Suzuki coupling.

Also provided is a method for making an advanced PBD intermediate from a compound of formula I, wherein R₂ is a triflate, including the steps of performing a C—C bond forming reaction on the triflate, and optionally, deprotecting the amine.

In some embodiments of this method, the C—C bond forming reaction is Suzuki coupling.

In some embodiments of these methods, when the amine is deprotected, the method includes a further step of coupling a linker to the deprotected amine.

### BRIEF DESCRIPTION OF THE DRAWINGS

FIG. 1 provides the structures of two PBD intermediates described herein. FIG. 1A shows PBD Core A and FIG. 1B shows PBD Core A triflate.

FIG. 2 provides a scheme for the synthesis of PBD Core A from commercially available starting materials.

FIG. 3 provides a scheme for the elaboration of PBD Core A through an advanced PBD intermediate to a PBD fragment. FIG. 3A highlights the synthesis of Fragment A through the Common Intermediate disclosed herein; FIG. 3B highlights the synthesis of Fragment B through the Common Intermediate.

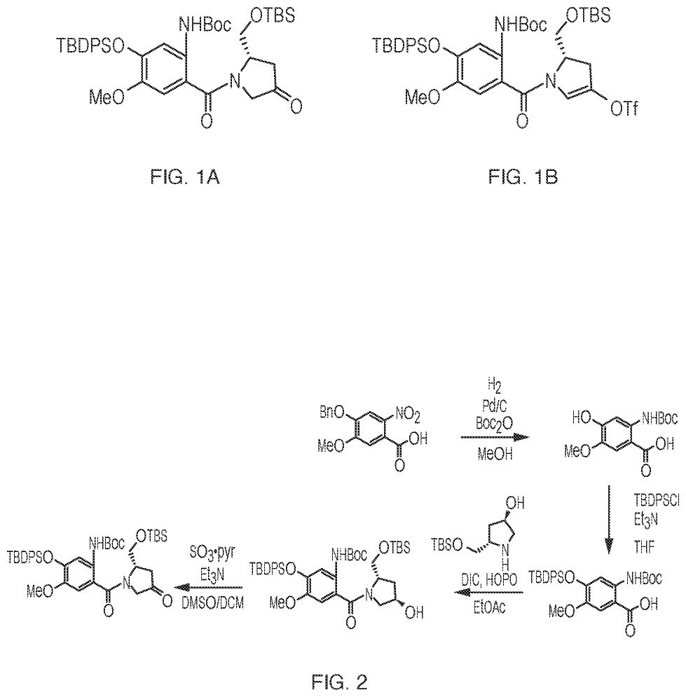

### DETAILED DESCRIPTION

New pyrrolobenzodiazepine (PBD) core and advanced intermediates are provided. These compounds are particularly amenable to the synthesis of PBD payloads used in antibody-drug conjugates. These compounds reduce the number of steps needed to synthesize PBD payloads, simplifying the process. These PBD intermediates provide considerable utility for developers interested in pursuing multiple analogs of PBD payloads due to the synthetic flexibility offered.

The PBD intermediates and methods provided herein solve the problem of drug investigators needing to develop lengthy de novo syntheses to arrive at individual PBD's payloads. The PBD intermediates provided herein can be used to prepare many of the PBD-derived payloads that have entered the clinic.

Representative PBD intermediate described herein include PBD Core A and PBD Core A triflate:

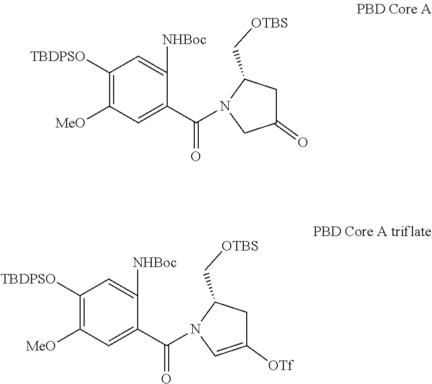

One feature of the PBD intermediates described herein is the Boc-protected nitrogen versus the commonly utilized nitro-group. The Boc-group offers versatility in synthetic choices in downstream chemistry. It provides the opportunity to prepare novel PBD's that are not tolerant of the nitro-reduction conditions. Moreover, early reduction avoids hazardous zinc dust reduction otherwise necessary on advanced intermediate. Furthermore, the Boc-group may be selectively removed or carried through the route, offering greater flexibility.

Older methods utilize a —NO₂ group which must be reduced after a ketone functionalization step. The change in the order of chemical steps means that the sequence has greater substrate compatibility, reduces the number of steps post-differentiation and avoids the use of explosive zinc dust. Later stage introduction of R groups offers a later point of diversification which simplifies the sequence. The use of the Boc-protecting group allows for selective removal, if desired, or can be carried through the sequence and removed by a simple acidic deprotection in the final steps.

The ketone and triflate functionalities exemplified in PBD Core A and PBD Core A triflate provide further flexibility in preparing PBDs or advanced intermediates. The ketone of PBD Core A is amenable to ketone functionalization. The triflate group of PBD Core A triflate is highly regioselective. The elaboration from PBD Core A to PBD Core A triflate is high yielding and provides excellent selectivity for the desired enol triflate stereoisomer.

In one embodiment, the PBD intermediates are those of formula I:

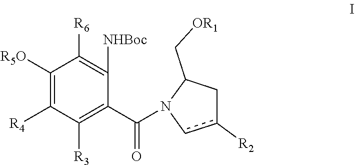

wherein the broken line represents a single or double bond. R₁ and R₅ are each alcohol protecting groups and are chosen such that each protecting group may be removed separately. In other words, R₁ and R₅ are selected to be orthogonal to each other. In some embodiments, R₁ is tert-butyldimethylsilyl ether (TBS). In other embodiments, R₁ is acetate (Ac). In some embodiments, R₅ is tert-butyldiphenylsilyl ether (TBDPS) and in other embodiments, triisopropylsilyl ether (TIPS). In a preferred embodiment, R₁ is TBS and R₅ is TBDPS.

R₂ may be selected from —H, ═O, —OTf (trifluoromethanesulfonate functional group, CF₃, SO₃—, or trifate), —OH, alkyl, cycloalkyl, alkenyl, aryl, heteroaryl, and a linker. Optionally, R₂ may be substituted with one or more substituents selected from halo, nitro, cyano, ether, carboxy, ester, alkyl, alkenyl, heterocyclyl, bis-oxy-C₁-C₃ alkylene and a linker.

In some embodiments, R₂ may be selected from —H, ═O, —OTf, —OH, C₁-C₅ alkyl, C₃-C₆ cycloalkyl, C₁-C₅ alkenyl, C₅-C₁₀ aryl, C₅-C₁₀ heteroaryl, and a linker. Optionally, R₂ may be substituted with one or more substituents selected from halo, nitro, cyano, ether, carboxy, ester, C₁-C₇ alkyl, C₁-C₇ alkenyl, C₃-C₇ heterocyclyl, bis-oxy-C₁-C₃ alkylene and a linker.

R₃ is selected from —H, —OH, —OR, —SH, —NH₂, —NO₂, halo, wherein R is selected from alkyl, heterocyclyl and aryl. R may optionally be substituted with an alkyl or aryl group.

In some embodiments, R₃ is selected from —H, —OH, —OR, —SH, —NH₂, —NO₂, halo, wherein R is selected from C₁-C₄ alkyl, C₃-C₁₀ heterocyclyl and C₅-C₁₀ aryl. R may optionally be substituted with a C₁-C₄ alkyl or phenyl.

R₄ is selected from —H, —OH, —OR, —SH, —SR, —NH₂, —NHR, —NHRR′, nitro and halo, wherein R and R′ are independently selected from alkyl, heterocyclyl and aryl. R and R′ may optionally have a substituent selected from alkyl and aryl.

In some embodiments, R₄ is selected from —H, —OH, —OR, —SH, —SR, —NH₂, —NHR, —NHRR′, nitro and halo, wherein R and R′ are independently selected from C₁-C₄ alkyl, C₃-C₁₀ heterocyclyl and C₅-C₁₀ aryl. R and R′ may optionally have a substituent selected from C₁-C₄ alkyl and phenyl.

R₆ is selected from —H, —OH, —OR, —SH, —NH₂, —NO₂, halo, wherein R is selected from alkyl, heterocyclyl and aryl, and R may optionally have a substituent selected from alkyl and aryl.

In some embodiments, R₆ is selected from —H, —OH, —OR, —SH, —NH₂, —NO₂, halo, wherein R is selected from C₁-C₄ alkyl, C₃-C₁₀ heterocyclyl and C₅-C₁₀ aryl, and R may optionally have a substituent selected from C₁-C₄ alkyl, phenyl.

In some embodiments, R₃ and R₆ are each —H.

In some embodiments, R₂ is selected from —H, ═O, —OTf, —OH, methyl, cyclopropyl, ═CH₂, ethylenyl, propenyl, phenyl, 4-fluorophenyl, indole and a linker. R₂ may be optionally substituted with one or more substituents selected from halo, nitro, cyano, ether, carboxy, ester, C₁-C₇ alkyl, C₁-C₇ alkenyl, C₃-C₇ heterocyclyl, bis-oxy-C₁-C₃ alkylene and a linker. In some embodiments R₂ is ═O and in some embodiments, R₂ is —OTf.

In some embodiments, R₄ is selected from —H, —OCH₃, —OCH₂Ph, —N(CH₃)₂, morpholino, piperidinyl, and —N—Me-piperazinyl. In some embodiments, R₄ is OCH₃.

In embodiments including linkers, the suitable linkers include suitable for use in ADCs. Such linkers may include, for example, cleavable linkers or non-cleavable linkers. The choice of linker will depend on such factors as point of attachment to the PBD Core intermediate, to specific antibody that will ultimately be used, to the desired point of release at the target. Suitable linkers may be identified by those skilled in the art. In some embodiments, the linker includes a 4-aminobenzyl (PAB) group attached to a dipeptide, such as valine-citrulline.

In some embodiments, the compound of formula I is one of

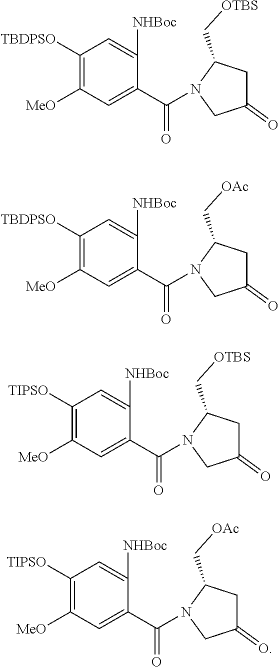

In other embodiments, the compound of formula I is one of

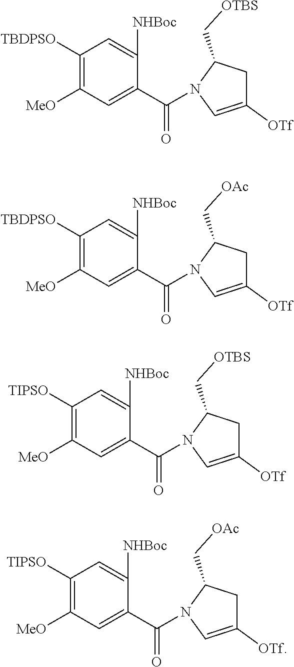

In still other embodiments, the compound of formula I may be:

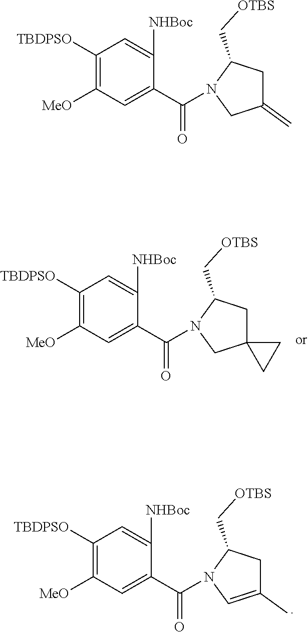

Also provided are analogues of formula I in which one or more of the protecting groups have been removed. Exemplary compounds include:

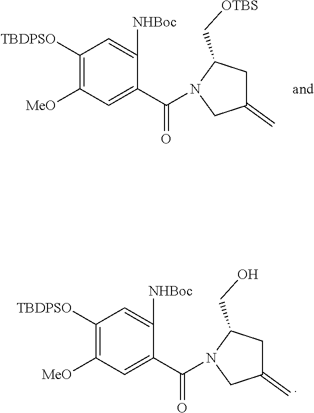

In certain other embodiments, compounds of formula II are provided:

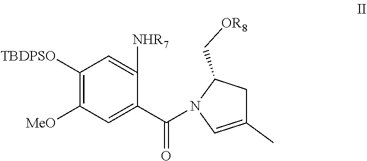

wherein R₇ is selected from —H, —C(O)O-TBS, Alloc, and linkers, such as protected PABC-peptide linkers, and R₅ is selected from —H and TBS. Exemplary compounds of formula II include:

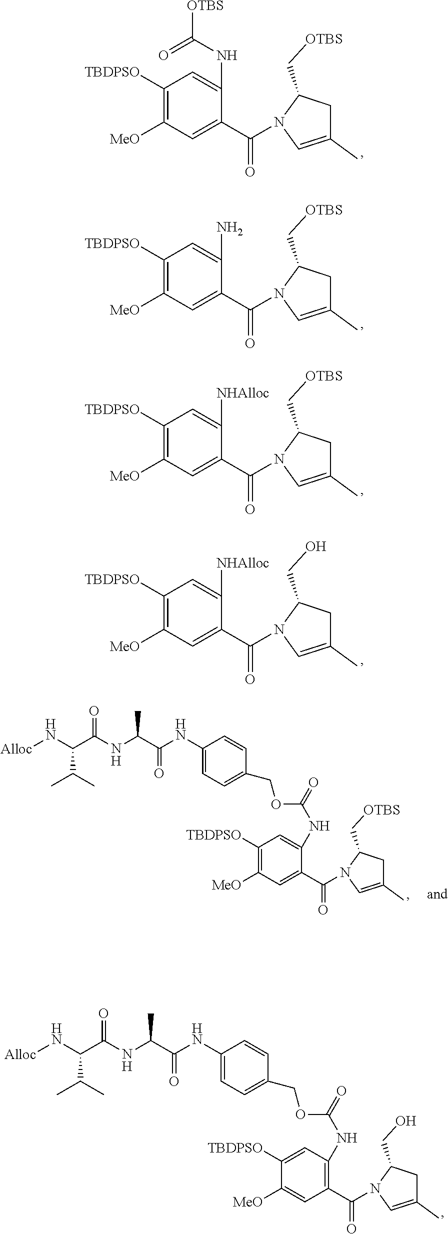

Also provided are intermediates of formula III:

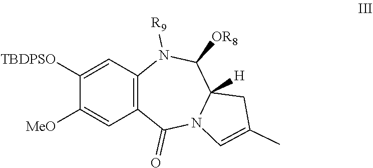

wherein R₉ is selected from Alloc and a linker, such as protected PABC-peptide linkers, and R₅ is selected from —H and TBS. Exemplary compounds of formula III include:

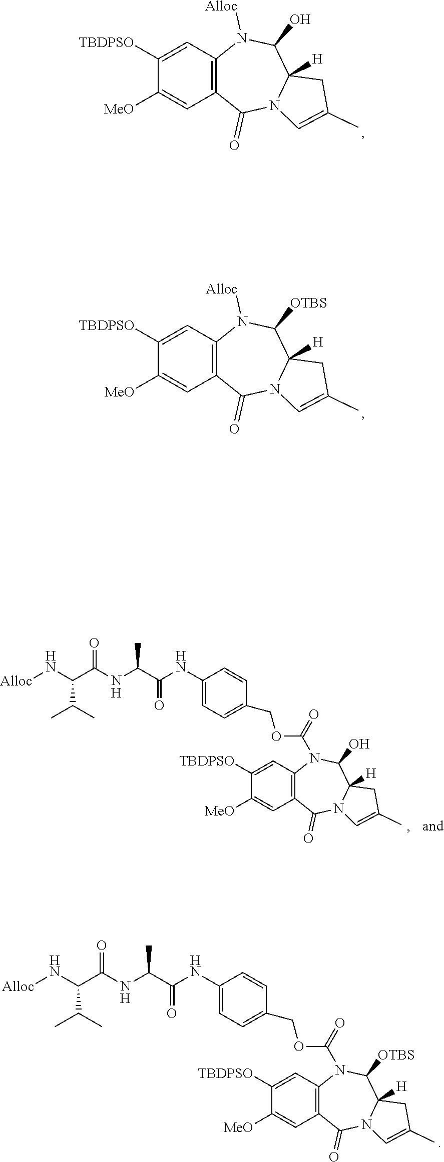

The intermediates provided herein may be prepared using commercially available starting materials as described below.

Also provided are methods for making an advanced PBD intermediate, the methods including further reactions at R₂. In particular, the presence of a ketone or triflate group at R₂ is amenable to carbon-carbon cross-coupling reactions. Many carbon-carbon cross-coupling reactions, or C—C cross-coupling reactions, are known to those skilled in the art. Some exemplary C—C cross-coupling reactions include, but are not limited to, Suzuki-Miyaura coupling or Suzuki coupling, Wittig reactions, aldol reactions, a-C—H arylations, Heck coupling, Negishi coupling, Stille coupling, and so forth. In accordance with the methods provided herein, a number of different C—C cross-coupling reactions are suitable. In some embodiments, Suzuki coupling is a preferred method of C—C cross-coupling.

In one embodiment, the method includes the steps of providing a compound of formula I wherein R₂ is a ketone, performing a C—C bond forming reaction at the ketone, and optionally, deprotecting the amine.

In some embodiments, the method includes the step of converting the ketone to a triflate, then performing a C—C bond forming reaction on the triflate, and optionally, deprotecting the amine.

In some embodiments, the C—C bond forming reaction is Suzuki coupling.

Also provided is a method for making an advanced PBD intermediate from a compound of formula I, wherein R₂ is a triflate, including the steps of performing a C—C bond forming reaction on the triflate, and optionally, deprotecting the amine.

In some embodiments of this method, the C—C bond forming reaction is Suzuki coupling.

In some embodiments of these methods, when the amine is deprotected, the method includes a further step of coupling a linker to the deprotected amine.

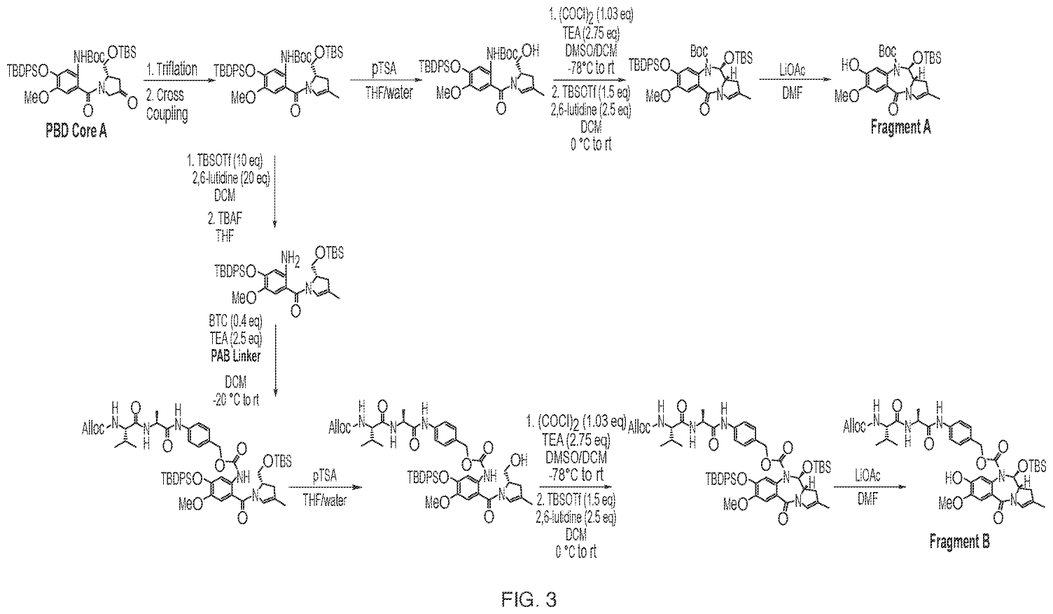

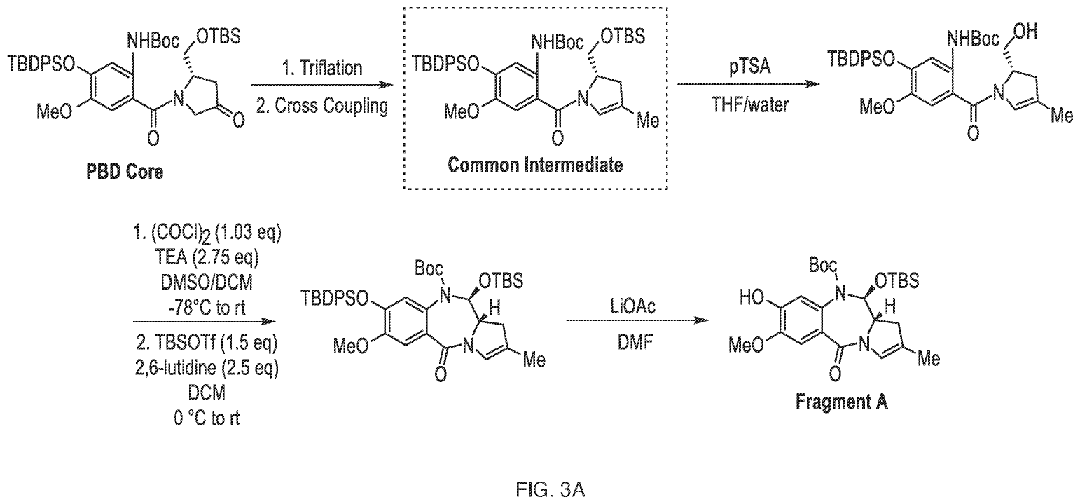

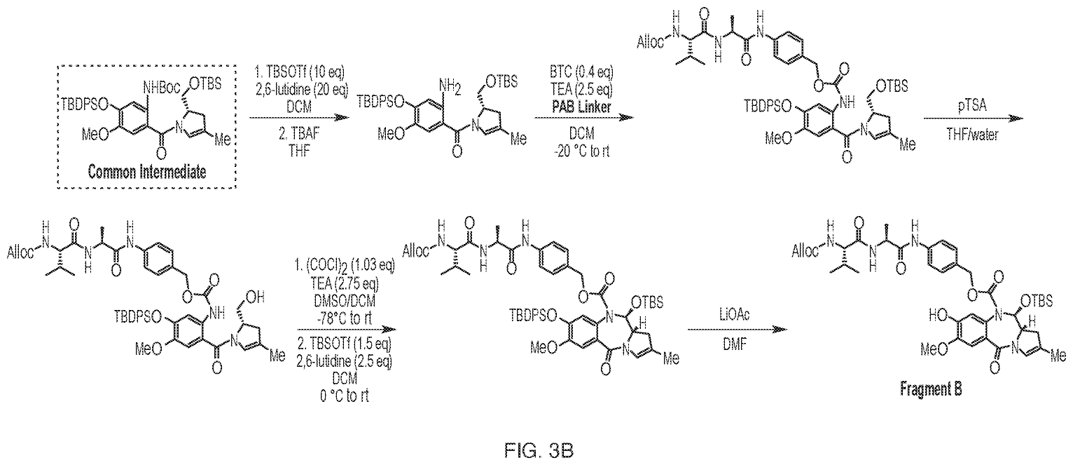

The following intermediates and advanced intermediates have been prepared as outlined below:

| Intermediate | Structure | |
|---|---|---|
| 3 | 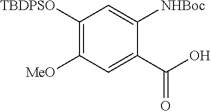 | PBD Benzoic Acid |
| 5 | 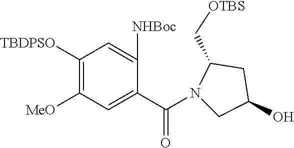 | Used in the preparation of PBD Core A |
| 6 | 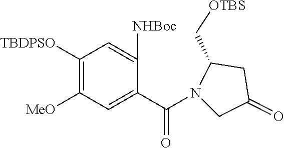 | PBD Core A |
| 7 | 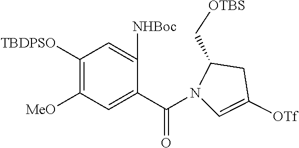 | PBD Core A triflate, PBD vinyl triflate |
| 8 | 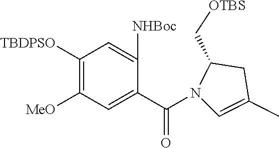 | Common Intermediate (FIG. 3) |
| 9 | 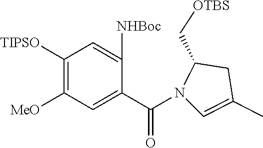 | |
| 10 | 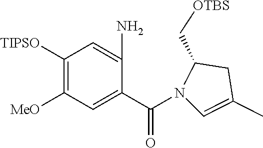 | |
| 11 | 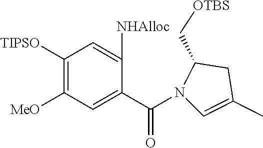 | |
| 12 | 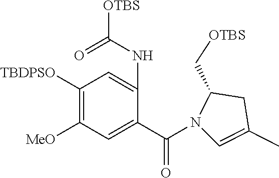 | |
| 13 | 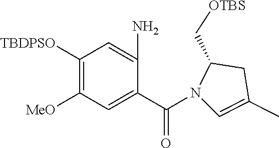 | |
| 14 | 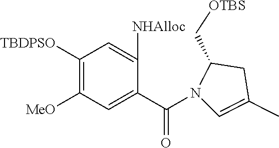 | |
| 15 | 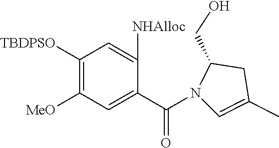 | |
| 16 | 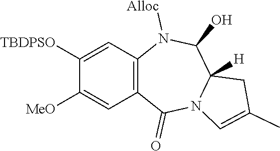 | |
| 17 | 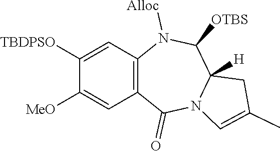 | |
| 18 | 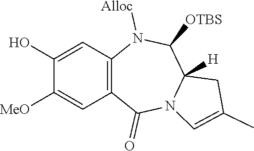 | Fragment A |
| 19 | 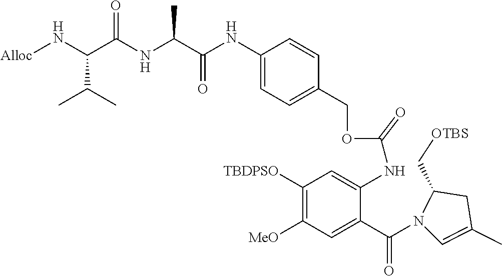 | |
| 20 | 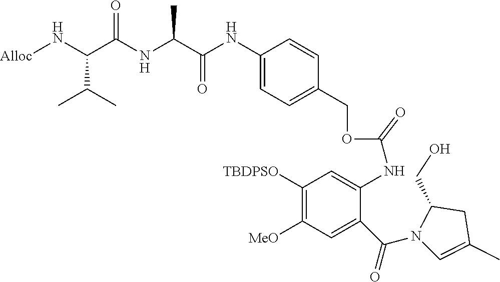 | |
| 21 | 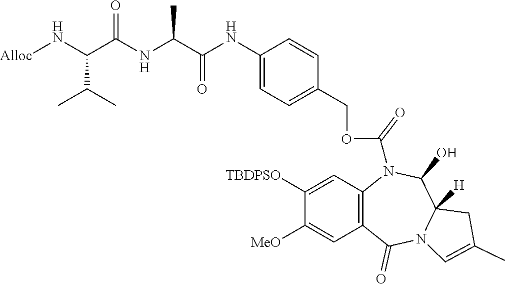 | |
| 22 | 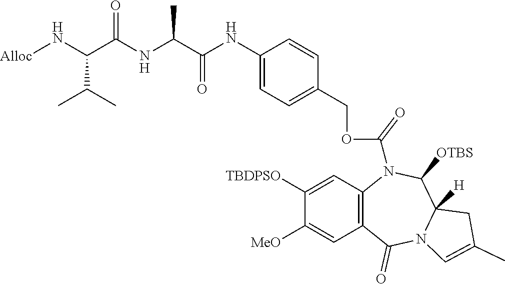 | |
| 23 | 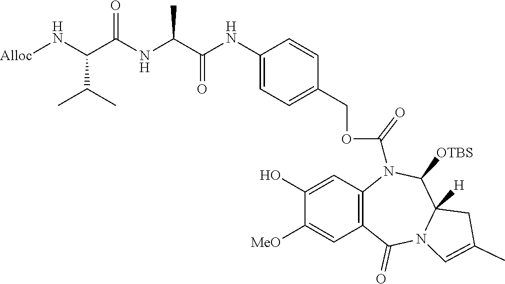 | |
| 24 | 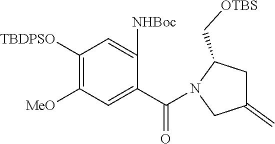 | |
| 25 | 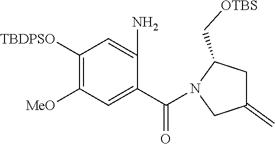 | |
| 26 | 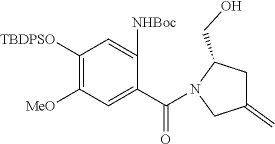 | |
| 27 | 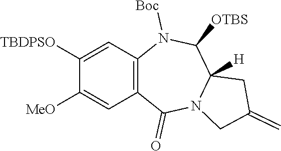 | |
| 28 | 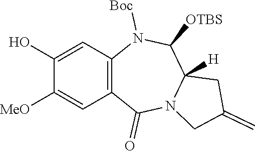 [SMILES: C=C1C[C@H]2[C@H](O[Si](C)(C)C(C)(C)C)N(C(=O)OC(C)(C)C)c3cc(O)c(OC)cc3C(=O)N2C1] | |

The following examples illustrate the preparation and use of the intermediates described herein.

### Example 1. Preparation of PBD Core A

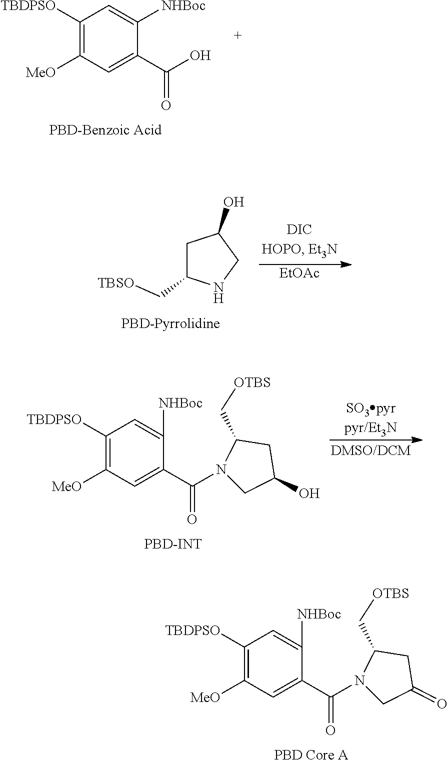

tert-butyl-(2-((2S,4R)-2-(((tert-butyldimethylsilyl)oxy)methyl)-4-hydroxypyrrolidine-1-carb-onyl)-5-((tert-butyldiphenylsilyl)oxy)-4-methoxyphenyl) carbamate: A solution of PBD-Benzoic Acid (5.0 g, 9.58 mmol, 1.0 equiv) in ethyl acetate (10 vol) was cooled to 0° C. The solution was charged with 2-hydroxypyridine 1-oxide (HOPO, 1.28 g, 11.5 mmol, 1.2 equiv) followed by DIC (1.78 mL, 4.79 mmol, 1.2 equiv) at such a rate to keep the temperature below 5° C. The reaction mixture was stirred for 30 min, then charged with PBD-pyrrolidine (2.2 g, 9.58 mmol, 1.0 equiv) in two portions. The mixture was then warmed to 20° C. and stirred for 3.5 h. When complete by HPLC′, the mixture was filtered through a pad of celite. The filtrate was then washed once with water (10 vol), and the aqueous layer was extracted with ethyl acetate (5 vol). The organic layers were combined and washed twice with 1M HCl (2×5 vol) followed by 1M sodium bicarbonate (5 vol) and 2M brine (5 vol). A solvent swap to heptane (5 vol) via constant volume distillation was performed, and the resulting precipitate was removed by filtering over celite. The filtrate was concentrated in vacuo to provide the PBD-INT as an off-white solid, which was used crude in the next step (80-90% yield).

tert-butyl-(S)-(2-(2-(((tert-butyldimethylsilyl)oxy)methyl)-4-oxopyrrolidine-1-carbonyl)-5-((tert-butyldiphenylsilyl)oxy)-4-methoxyphenyl) carbamate: A solution of sulfur trioxide-pyridine complex (7.07 g, 44.4 mmol, 6.0 equiv) in DMSO (10 vol) was prepared and stirred for 20 min. The sulfur trioxide-pyridine solution was charged with pyridine (3.59 mL, 44.4 mmol, 6.0 equiv) and stirred for an additional 30 min. In a separate flask, a solution of crude PBD-INT (5.44 g, 7.40 mmol, 1.0 equiv) in 10 vol of DCM was prepared and cooled to 0° C. The chilled solution was charged with triethylamine (8.25 mL, 59.2 mmol, 8.0 equiv). The sulfur trioxide-pyridine solution was added to the cooled solution of PBD-INT at such a rate to keep the temperature below 10° C. The reaction mixture was warmed to 20° C. and stirred for 3 h. When the reaction was complete by HPLC, the mixture was cooled to 0° C. and quenched with 1M sodium bicarbonate (5 vol). The organic phase was collected, and the aqueous phase was extracted with 5 vol of DCM. The organic phases were combined and washed three times with 1M brine (3×5 vol). The organic layer was concentrated in vacuo and the product was purified via column chromatography (12-22% ethyl acetate/heptane). The product-containing fractions were combined and isolated to afford PBD Core A (>95% purity, 75% yield).

### Example 2. Triflation/Suzuki coupling/Boc-deprotection

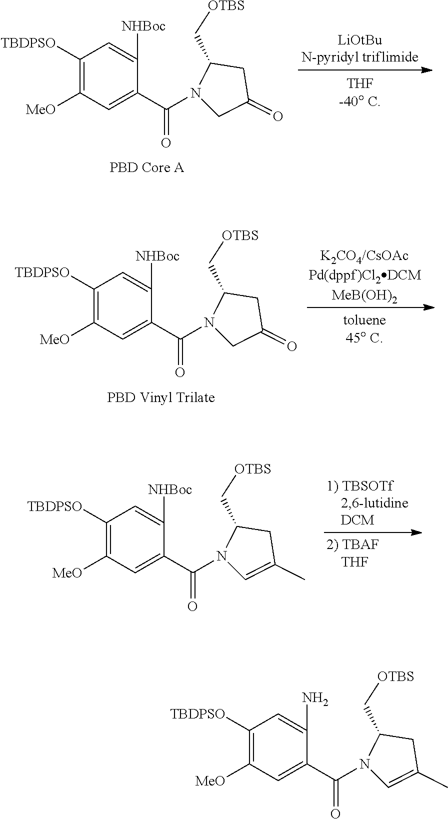

(S)-1-(2-((tert-butoxycarbonyl)amino)-4-((tert-butyldiphenylsilyl)oxy)-5-methoxybenzoyl)-5-(((tert-butyldimethylsilyl)oxy)methyl)-4,5-dihydro-1H-pyrrol-3-yl trifluoromethanesulfonate. A solution of PBD Core A (500.0 mg, 0.682 mmol, 1.0 equiv) in THF (40 mL) was prepared under a nitrogen atmosphere. The solution was charged with N-(2-pyridyl)bis(trifluoromethanesulfonimide) (489.0 mg, 1.36 mmol, 2.0 equiv) and cooled to −40° C. The solution was then charged with lithium tert-butoxide (1M solution in THF, 1.71 mL, 1.71 mmol, 2.50 equiv) in one portion. The reaction was stirred at −40° C. until complete by HPLC (2.5h). The reaction was quenched with 10% citric acid in ethyl acetate (40 vol), and then warmed to 0° C. The reaction mixture was charged with 40 vol of water, and the organic layer was collected and washed with 2M brine (10 vol), dried over sodium sulfate, and concentrated in vacuo. The residue was taken up in heptane (5 vol) and filtered through celite. The filtrate was concentrated in vacuo and purified via column chromatography (0-10% ethyl acetate/heptane). The product-containing fractions were combined and concentrated in vacuo to afford the vinyl triflate (75-80%).

(S)-1-(2-((tert-butoxycarbonyl)amino)-4-((tert-butyldiphenylsilyl)oxy)-5-methoxybenzoyl)-5-(((tert-butyldimethylsilyl)oxy)methyl)-4,5-dihydro-1H-pyrrol-3-yltrifluoromethanesulfonate: A suspension of potassium phosphate, tribasic (5.8 g, 27 mmol, 5.0 equiv) and cesium acetate (1.0 g, 5.5 mmol, 1.0 equiv) was prepared in degassed toluene (6.0 vol) under nitrogen. The suspension was heated to 45° C. and Pd (dppf) Cl₂.DCM was added to the suspension. In a separate flask, a solution of the PBD Vinyl Triflate (4.7 g, 5.5 mmol, 1.0 equiv) in degassed toluene (6.0 vol) was prepared under nitrogen. The vinyl triflate solution was added dropwise to the heated suspension. Once the addition of the vinyl triflate was complete, methyl boronic acid (1.3 g, 22 mmol, 4.0 equiv) was added portionwise. The reaction was stirred at 45° C. until complete by HPLC (typically 1.5 h). The mixture was then cooled to room temperature and poured into water (12 vol). The organic phase was collected, concentrated in vacuo, and purified via column chromatography (0-15% ethyl acetate/heptane). The product-containing fractions were combined and isolated to afford the product (30-50% yield).

(S)-(2-amino-4-((tert-butyldiphenylsilyl)oxy)-5-methoxyphenyl) (2-(((tert-butyldimethylsilyl)oxy)methyl)-4-methyl-2,3-dihydro-1H-pyrrol-1-yl) methanone: A solution of the Suzuki product (230.0 mg, 0.315 mmol, 1.0 equiv) in DCM (10 vol) was prepared. The solution was charged with 2,6-lutidine (0.729 mL, 6.29 mmol, 20.0 equiv) followed by TBSOTf (0.722 mL, 3.15 mmol, 10.0 equiv). The solution was then heated to 40° C. and stirred for 15 h. When complete by HPLC, the mixture was quenched with sat. ammonium chloride (10 vol). The aqueous layer was extracted with 10 vol of DCM, and the organic layers were combined, washed with 2M brine (10 vol), dried over magnesium sulfate, and then concentrated in vacuo. The residue was taken up in THF (10 vol) and cooled to 0° C. The solution was charged with TBAF (1M solution in THF, 0.157 mL, 0.16 mmol, 0.5 equiv) and stirred at 0° C. for 30 min. The solution was then diluted with 10 vol of ethyl acetate and quenched with 10 vol of water. The layers were separated, and the aqueous layer was extracted with ethyl acetate (10 vol). The organic layers were combined, dried over magnesium sulfate, and concentrated in vacuo. The product was purified via column chromatography (15% ethyl acetate/heptane). The product-containing fractions were combined and isolated to afford the free aniline (40-60% yield).

### Example 3 Preparation of PBD Benzoic Acid

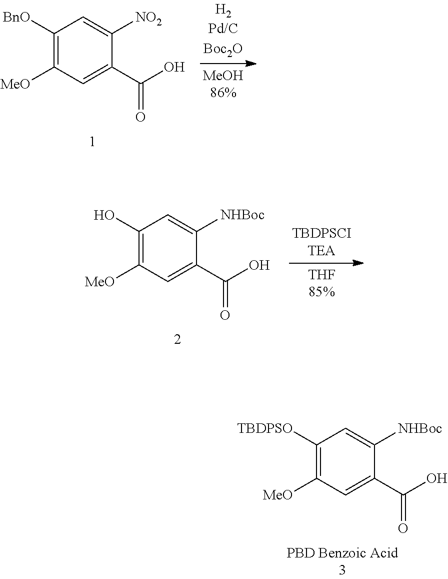

2-((tert-butoxycarbonyl)amino)-4-hydroxy-5-methoxybenzoic acid (2): Benzoic acid 1 (1.0 equiv) was dissolved in 15 vol of MeOH. The resulting mixture was charged with 10% palladium on carbon (0.03 equiv, 10% w/w) and BoczO (5 equiv). The reaction vessel was evacuated and back-filled with nitrogen 3 times. The reaction vessel was equipped with a hydrogen-filled balloon, and it was then evacuated and back-filled with hydrogen 3 times. The reaction was stirred overnight until complete conversion to 2 was observed by TLC. After the reaction was complete, the mixture was solvent swapped to hexane (10 vol) and stirred for 1 h at room temperature. The residue was then filtered over celite. The filtrate was concentrated in vacuo to afford the final product as light brown to pink solid in 86% yield. ¹H NMR (400 MHZ, DMSO-d6) δ 13.10 (br s, 1H), 10.49 (s, 1H), 10.15 (br s, 1H), 7.88 (s, 1H), 7.39 (s, 1H), 3.74 (s, 3H) 1.47 (s, 9H); HRMS: calculated for C₁₃H₁₆NO₆—: 282.0983 found: 282.0984.

2-((tert-butoxycarbonyl)amino)-4-hydroxy-5-methoxybenzoic acid: A solution of 2 (1.1 equiv) in THF (20 vol) was charged with triethylamine (8 eq.). The mixture was stirred for 30 min. The solution was then charged with tert-butyl(chloro) diphenyl silane (3.0 equiv) slowly. The reaction was stirred at room temperature for 5.5 h. The reaction was quenched with 1M HCl (12 vol), and the mixture was concentrated in vacuo to remove the THF. The mixture was then charged with ethyl acetate (15 vol). The aqueous layer was back extracted with ethyl acetate (5 vol) and the organic layers phases were combined and washed with 2M brine (5 vol). The aqueous layer was back extracted with ethyl acetate (5 vol.), and the organic phases were combined and concentrated in vacuo. The crude product was then charged with 15% MTBE/85% heptane and the resulting slurry was heated to 50° C. The resulting solution was stirred at 50° C. for 1.5h, then cooled to room temperature and stirred at room temperature for 2 h. The material was filtered to isolated PBD-Benzoic acid as a light brown to off-white solid (57% yield, 97% purity). The solid was then dissolved in MTBE (10 vol) and the solution was charged with CUNO Type 2 activated charcoal (10% w/w). The mixture was stirred for 2 h, then filtered through celite. The filtrate was concentrated in vacuo to provide the product as a white foam (88% recovery, 99.5% purity). ¹H NMR (400 MHZ, DMSO-d6) δ 13.30 (br s, 1H), 10.30 (s, 1H), 7.86 (s, 1H), 7.64 (dd, J=1.47, 7.82 Hz, 4H), 7.36-7.47 (m, 6H), 7.32-7.36 (s, 1H), 3.39 (s, 3H), 1.39 (s, 9H), 1.05 (s, 9H); HRMS: calculated for C₂₉H₃₄NO₆Si—: 520.2161 found: 520.2110.

### Example 4. Preparation of PBD Core

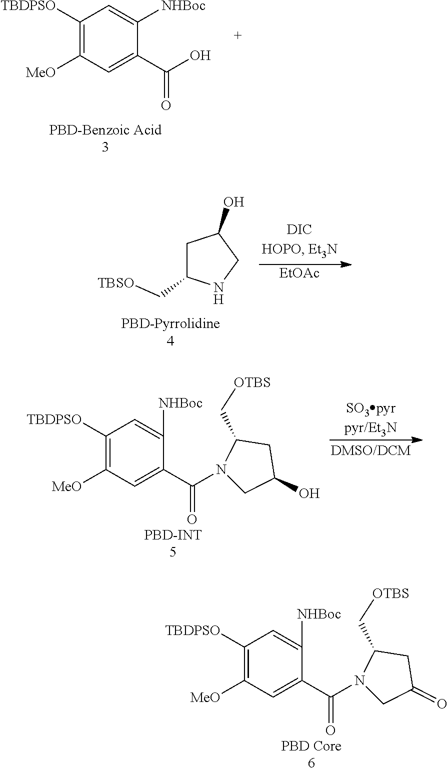

tert-butyl-(2-((2S,4R)-2-(((tert-butyldimethylsilyl)oxy)methyl)-4-hydroxypyrrolidine-1-carb-onyl)-5-((tert-butyldiphenylsilyl)oxy)-4-methoxyphenyl) carbamate: A solution of PBD-Benzoic Acid (5.0 g, 9.58 mmol, 1.0 equiv) in ethyl acetate (10 vol) was cooled to 0° C. The solution was charged with 2-hydroxypyridine 1-oxide (HOPO, 1.28 g, 11.5 mmol, 1.2 equiv) followed by DIC (1.78 mL, 4.79 mmol, 1.2 equiv) at such a rate to keep the temperature below 5° C. The reaction mixture was stirred for 30 min, then charged with PBD-pyrrolidine (2.2 g, 9.58 mmol, 1.0 equiv) in two portions. The mixture was then warmed to 20° C. and stirred for 3.5 h. When complete by HPLC, the mixture was filtered through a pad of celite. The filtrate was then washed once with water (10 vol), and the aqueous layer was extracted with ethyl acetate (5 vol). The organic layers were combined and washed twice with 1M HCl (2×5 vol) followed by 1M sodium bicarbonate (5 vol) and 2M brine (5 vol). A solvent swap to heptane (5 vol) via constant volume distillation was performed, and the resulting precipitate was removed by filtering over celite. The filtrate was concentrated in vacuo to provide the PBD-INT as an off-white solid, which was used crude in the next step (80-90% yield). ¹H NMR (400 MHZ, DMSO-d6) δ 8.85 (s, 1H), 7.65 (ddd, J=7.86, 3.64, 1.47 Hz, 4H), 7.36-7.49 (m, 7H), 6.70 (s, 1H), 4.94-4.82 (m, 1H), 4.28 (m, 1H), 4.12-4.22 (m, 1H), 3.85-3.67 (m, 2H), 3.52-3.43 (m, 1H), 3.38 (s, 3H), 3.21 (br d, J=10.76 Hz, 1H), 2.07-1.85 (m, 2H), 1.37 (s, 9H), 1.08-1.05 (m, 9H), 0.90-0.81 (m, 9H), 0.06-0.02 (m, 6H); HRMS: calculated for C₄₀H₅₉N₂O₇Si₂⁺: 735.3855 found: 735.3864.

tert-butyl-(S)-(2-(2-(((tert-butyldimethylsilyl)oxy)methyl)-4-oxopyrrolidine-1-carbonyl)-5-((tert-butyldiphenylsilyl)oxy)-4-methoxyphenyl) carbamate: A solution of sulfur trioxide-pyridine complex (7.07 g, 44.4 mmol, 6.0 equiv) in DMSO (10 vol) was prepared and stirred for 20 min. The sulfur trioxide-pyridine solution was charged with pyridine (3.59 mL, 44.4 mmol, 6.0 equiv) and stirred for an additional 30 min. In a separate flask, a solution of crude PBD-INT (5.44 g, 7.40 mmol, 1.0 equiv) in 10 vol of DCM was prepared and cooled to 0° C. The chilled solution was charged with triethylamine (8.25 mL, 59.2 mmol, 8.0 equiv). The sulfur trioxide-pyridine solution was added to the cooled solution of PBD-INT at such a rate to keep the temperature below 10° C. The reaction mixture was warmed to 20° C. and stirred for 3 h. When the reaction was complete by HPLC, the mixture was cooled to 0° C. and quenched with 1M sodium bicarbonate (5 vol). The organic phase was collected, and the aqueous phase was extracted with 5 vol of DCM. The organic phases were combined and washed three times with 1M brine (3×5 vol). The organic layer was concentrated in vacuo and the product was purified via column chromatography (12-22% ethyl acetate/heptane). The product-containing fractions were combined and isolated to afford PBD Core A (>95% purity, 75% yield). ¹H NMR (400 MHZ, DMSO-d6) δ 8.41 (s, 1H), 7.68-7.62 (m, 4H), 7.45-7.34 (m, 7H), 7.15 (s, 1H), 6.74 (s, 1H), 4.51-4.73 (s, 1H), 3.91-3.77 (m, 1H), 3.69 (s, 1H), 3.67-3.58 (m, 1H), 3.44 (m, 3H), 2.87-2.83 (dd, J=17.52, 9.16 Hz, 1H), 2.52-2.46 (d, J=17.72, 1H), 1.35 (s, 9H), 1.08 (s, 9H), 0.82 (s, 9H), 0.00 (s, 6H); 13C NMR (400 MHZ, DMSO-d6) δ 210.1, 168.6, 153.2, 146.6, 146.1, 135.4, 133.2, 130.27, 128.03, 115.5, 111.9, 79.6, 56.0, 31.7, 28.5, 27.0, 26.0, 22.5, 19.8 18.2, 14.2,-5.4; IR (Neat) 3344, 3073, 2954, 2930, 2896, 2857, 1765, 1726, 1627, 1589, 1516, 1471, 1427, 1391, 1366, 1251, 1238, 1155, 1106, 1013, 942, 874, 834, 776, 700; HRMS: calculated for C₄₀H₅₇N₂O₇Si₂⁺: 733.3699 found: 733.3700.

### Examples 5-8 Downstream Chemistry

### Example 5. Preparation of PBD Vinyl Triflate

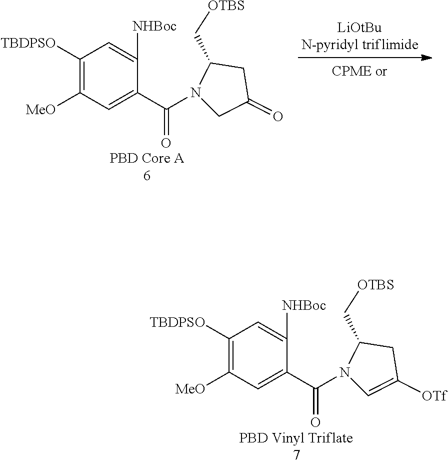

(S)-1-(2-((tert-butoxycarbonyl)amino)-4-((tert-butyldiphenylsilyl)oxy)-5-methoxybenzoyl)-5-(((tert-butyldimethylsilyl)oxy)methyl)-4,5-dihydro-1H-pyrrol-3-yl trifluoromethanesulfonate: A solution of PBD Core (1.0 equiv) in CPME or THF (40 vol) was prepared under a nitrogen atmosphere. The solution was charged with N-(2-pyridyl)bis(trifluoromethanesulfonimide) (1.2 equiv) and cooled to −40° C. The solution was then charged with lithium tert-butoxide (1M solution in THF, 1.71 mL, 1.71 mmol, 2.50 equiv) in one portion. The reaction was stirred at −40° C. until complete by HPLC (2.5h). The reaction was quenched with 10% citric acid in ethyl acetate (40 vol), and then warmed to 0° C. The reaction mixture was charged with 40 vol of water, and the organic layer was collected and washed with 2M brine (10 vol), dried over sodium sulfate, and concentrated in vacuo. The residue was taken up in heptane (5 vol) and filtered through celite. The filtrate was concentrated in vacuo and purified via column chromatography (0-10% ethyl acetate/heptane). The product-containing fractions were combined and concentrated in vacuo to afford the vinyl triflate (75-80%). HRMS: calculated for C₄₁H₅₆F₃N₂O₉SSi₂⁺: 865.3192 found: 865.3157.

### Example 6. Cross Coupling Procedures

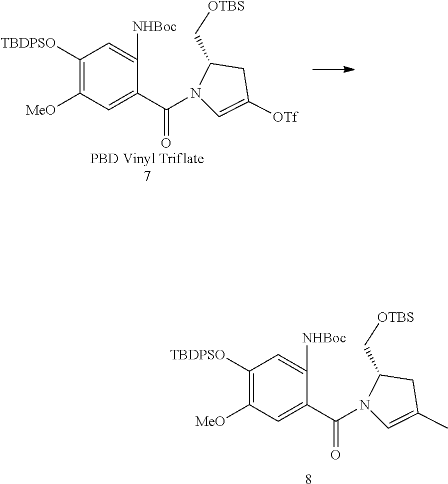

### Example 6A. Suzuki Coupling

A suspension of potassium phosphate, tribasic (5.0 equiv) and cesium acetate (1.0 equiv) was prepared in degassed toluene (6.0 vol) under nitrogen. The suspension was heated to 45° C. and Pd (dppf) Cl2·DCM was added to the suspension. In a separate flask, a solution of the PBD Vinyl Triflate (1.0 equiv) in degassed toluene (6.0 vol) was prepared under nitrogen. The vinyl triflate solution was added dropwise to the heated suspension. Once the addition of the vinyl triflate was complete, methyl boronic acid (4.0 equiv) was added portionwise. The reaction was stirred at 45° C. until complete by HPLC (typically 1.5 h). The mixture was then cooled to room temperature and poured into water (12 vol). The organic phase was collected, concentrated in vacuo, and purified via column chromatography (0-15% ethyl acetate/heptane). The product-containing fractions were combined and isolated to afford the product (30-50% yield).

### Example 6B. Copper Coupling

A suspension of copper (I) cyanide di(lithium chloride) complex (5.0 equiv) in THF or CPME (10 vol) was prepared and cooled to −40° C. The suspension was charged with methyl magnesium bromide (3.0M in diethyl ether, 10.0 equiv) dropwise. The mixture was warmed to 0° C. and stirred for 10 min then cooled back down to −40° C. A solution of the PBD Vinyl Triflate (1.0 equiv) in THF or CPME (10 vol) was added dropwise over 10 min. The reaction was stirred at −40° C. until complete by HPLC (typically 1 hr). Upon completion, the reaction was warmed to 0° C. and slowly quenched with saturated ammonium chloride (20 vol). The organic layer was collected and washed with saturated ammonium chloride (20 vol), saturated sodium bicarbonate (20 vol), and saturated brine (20 vol). The organic layer was collected solvent swapped to toluene (20 vol). The solution was then charged with SiliaMetS DOTA and stirred for 1 hr at room temperature. The mixture was filtered, and the filtrate was concentrated in vacuo to afford the crude product as a clear, yellow oil. The product was then purified via column chromatography (0-7% ethyl acetate/heptane). The product-containing fractions were combined and concentrated in vacuo to afford the product (40% yield).

tert-butyl(S)-(2-(2-(((tert-butyldimethylsilyl)oxy)methyl)-4-methyl-2,3-dihydro-1H-pyrrole-1-carbonyl)-5-((tert-butyldiphenylsilyl)oxy)-4-methoxyphenyl) carbamate (8) ¹H NMR (400 MHZ, CDCl3) δ 8.10 (br s, 1H), 7.75-7.62 (m, 5H), 7.42-7.27 (m, 7H), 6.53 (s, 1H), 6.04 (s, 1H), 4.60 (m, 1H), 3.97-3.67 (m, 2H), 3.18 (s, 3H), 2.76-2.61 (m, 1H), 2.56-2.45 (m, 1 H), 1.70-1.58 (m, 3H) 1.49-1.38 (m, 9H) 1.14-1.05 (m, 9H) 0.93-0.77 (m, 12H) 0.09-0.05 (m, 6H); 13C NMR (101 MHZ, CDCl3) δ 165.3, 152.7, 147.3, 144.9, 135.2, 129.4, 127.4, 125.4, 125.3, 123.6, 121.9, 113.7, 112.2, 79.86, 62.5, 59.1, 55.3, 36.3, 29.7, 28.3, 26.7, 25.7, 19.9, 18.1, 14.1, 13.7,. 01,-5.5; HRMS: calculated for C₄₁H₅₉N₂O₆Si₂⁺: 731.3906 found: 731.3960.

### Example 7. Formal Synthesis of Tesirine

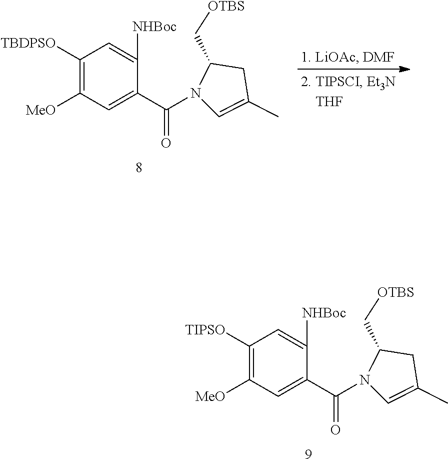

tert-butyl(S)-(2-(2-(((tert-butyldimethylsilyl)oxy)methyl)-4-methyl-2,3-dihydro-1H-pyrrole-1-carbonyl)-4-methoxy-5-((triisopropylsilyl)oxy)phenyl) carbamate (9): A solution of 8 (214 mg, 0.293 mmol, 1.0 equiv) in wet DMF (2 mL) was prepared. The solution was charged with lithium acetate (19.3 mg, 0.293 mmol, 1.0 equiv) and heated to 40° C. The mixture was stirred until complete by HPLC. The mixture was then cooled to room temperature and diluted with ethyl acetate (5 mL). The mixture was washed with water (5 mL). The aqueous layer was collected and back-extrated with ethyl acetate (5 mL). The organic layers were combined and washed with 5% citric acid (5 mL) followed by saturated brine (5 mL). The organic layer was dried over sodium sulfate, filtered, and concentrated in vacuo to afford the free phenol as a clear, light brown oil. The intermediate was dissolved in THF (2 mL) and cooled to 0° C. The solution was charged with triethylamine (81.6 μL, 0.585 mmol, 2.0 equiv) followed by TIPSCI (68.9 μL, 0.322 mmol, 2.0 equiv). The reaction mixture was allowed to reach room temperature and stirred for 15h. When the reaction was complete by HPLC, the mixture was filtered to remove the solids, and the solids were washed with THF (2 mL). The organic phases were combined and washed with saturated sodium bicarbonate (2 mL) and brine (2 mL). The organic phase was dried over sodium sulfate, filtered, and concentrated in vacuo. The crude product was purified via column chromatography (0-15% ethyl acetate/heptane), and the product containing fractions were combined and isolated to provide 9 (96.9 mg, 0.149 mmol, 51% yield over two steps). HRMS: calculated for C₃₄H₆₁N₂O₆Si₂⁺: 649.4063 found: 649.4049.

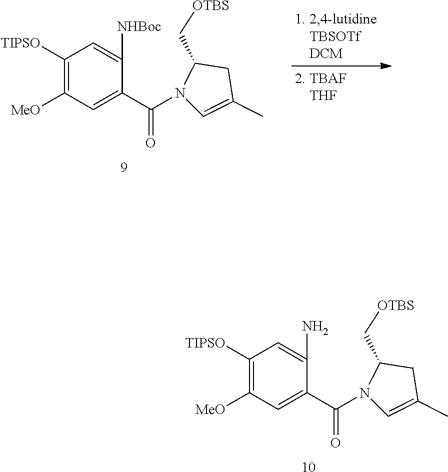

(S)-(2-amino-5-methoxy-4-((triisopropylsilyl)oxy)phenyl) (2-(((tert-butyldimethylsilyl)oxy)-methyl)-4-methyl-2,3-dihydro-1H-pyrrol-1-yl) methanone (10): A solution of 10 (96.9 mg, 0.149 mmol, 1.0 equiv) in DCM (10 vol) was prepared. The solution was charged with 2,6-lutidine (346 μL, 2.99 mmol, 20 equiv) and TBSOTf (343 μL, 1.49 mmol, 10.0 equiv). The solution was then heated to 40° C. and stirred for 15 h. When complete by HPLC, the mixture was quenched with sat. ammonium chloride (2 mL). The aqueous layer was back-extracted with DCM (2 mL), and the organic layers were combined, washed with saturated brine (2 mL), and dried over magnesium sulfate. The solution was concentrated in vacuo. The residue was taken up in THF (2 mL) and cooled to 0° C. The solution was charged with TBAF (14.9 μL 0.2 equiv) and stirred at 0° C. for 30 min. The solution was then diluted with 10 vol of ethyl acetate and quenched with 10 vol of water. The layers were separated, and the aqueous layer was extracted with ethyl acetate (10 vol). The organic layers were combined, dried over magnesium sulfate, and concentrated in vacuo. The product matched reported NMR data^{1b, 2} and was immediately used in the next step. HRMS: calculated for C₂₉H₅₃N₂O₄Si₂⁺: 549.3538 found: 549.3558.

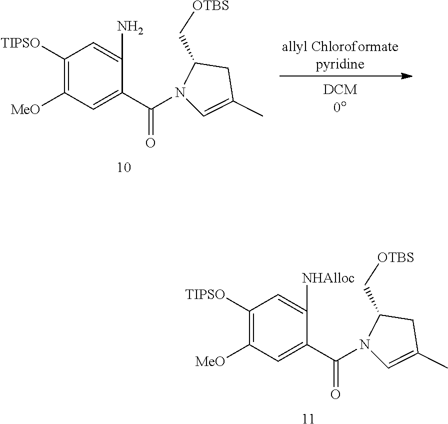

allyl(S)-(2-(2-(((tert-butyldimethylsilyl)oxy)methyl)-4-methyl-2,3-dihydro-1H-pyrrole-1-car-bonyl)-4-methoxy-5-((triisopropylsilyl)oxy)phenyl) carbamate: A solution of the free amine 10 (41 mg, 0.075 mmol, 1.0 equiv) in DCM (12 vol) was prepared and cooled to 0° C. The solution was charged with pyridine (13 μL, 0.16 mmol, 2.2 equiv) followed by allyl chloroformate (8.8 μL, .082 mmol, 1.1 equiv). The reaction was stirred at 0° C. until complete by HPLC (typically 1 hr). The reaction mixture was quenched with 10% citric acid solution (8 vol). The organic layer was collected and washed with saturated aqueous sodium bicarbonate (8 vol) and brine (8 vol). The organic phase was collected, dried over sodium sulfate, and the filtered. The solution was concentrated in vacuo to afford the crude product as a orange to brown oil. The product was purified via column chromatography (0-15% ethyl acetate/heptane), and the product containing fractions were combined and isolated to provide 11 (28 mg, 0.075 mmol, 59% yield over two steps). The isolated product matched reported NMR data. 1b, 2 HRMS: calculated for C₃₃H₅₇N₂O₆Si₂⁺: 633.3750 found: 633.3773.

### Example 8 Preparation of Advanced Intermediates from PBD Core

### Example 8A. General Procedure for Boc Deprotection

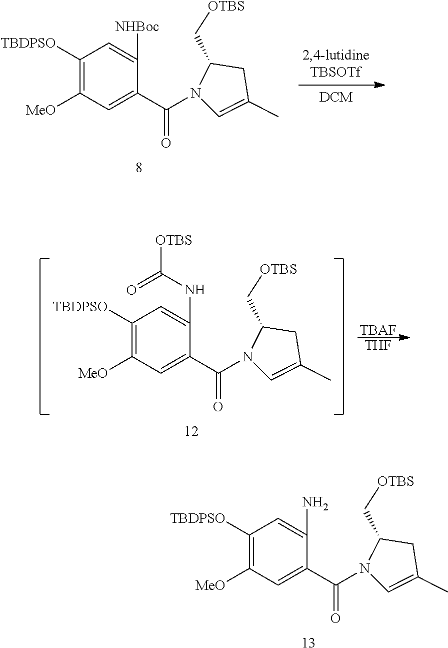

A solution of 8 (1.0 equiv) in DCM (10 vol) was prepared. The solution was charged with 2,6-lutidine (20 equiv) and TBSOTf (10.0 equiv). The solution was then heated to 40° C. and stirred for 15 h. When complete by HPLC, the mixture was quenched with sat. ammonium chloride (5 vol). The aqueous layer was back-extracted with DCM (5 vol), and the organic layers were combined, washed with saturated brine (5 vol), and dried over magnesium sulfate. The solution was concentrated in vacuo. The carbamate 12 (HRMS: calculated for C₄₃H₆₅N₂O₆Si₃⁺: 789.4145 found: 789.4115) was taken up in THF (10 vol) and cooled to 0° C. The solution was charged with TBAF (0.2 equiv) and stirred at 0° C. for 30 min. The solution was then diluted with 10 vol of ethyl acetate and quenched with 10 vol of water. The layers were separated, and the aqueous layer is extracted with ethyl acetate (10 vol). The organic layers were combined, dried over magnesium sulfate, and concentrated in vacuo to provide 13. The material was used immediately to avoid decomposition.

(S)-(2-amino-4-((tert-butyldiphenylsilyl)oxy)-5-methoxyphenyl) (2-(((tert-butyldimethylsilyl)oxy)methyl)-4-methyl-2,3-dihydro-1H-pyrrol-1-yl) methanone (13): HRMS: calculated for C₂₉H₅₃N₂O₄Si₂⁺: 549.3538 found: 549.3558.

### Example 8B

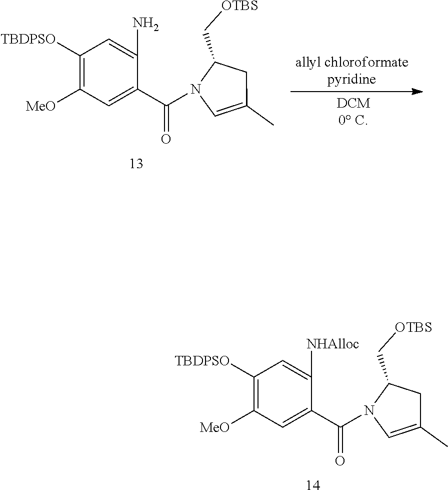

allyl(S)-(2-(2-(((tert-butyldimethylsilyl)oxy)methyl)-4-methyl-2,3-dihydro-1H-pyrrole-1-carbonyl)-5-((tert-butyldiphenylsilyl)oxy)-4-methoxyphenyl) carbamate (14): A solution of the free amine 13 (1.04 g, 1.69 mmol, 1.0 equiv) in DCM (12 vol) was prepared and cooled to 0° C. The solution was charged with pyridine (301 μL, 3.72 mmol, 2.2 equiv) followed by allyl chloroformate (198 μL, 1.86 mmol, 1.1 equiv). The reaction was stirred at 0° C. until complete by HPLC (typically 1 hr). The reaction mixture was quenched with 10% citric acid solution (8 vol). The organic layer was collected and washed with saturated aqueous sodium bicarbonate (8 vol) and brine (8 vol). The organic phase was collected, dried over sodium sulfate, and the filtered. The solution was concentrated in vacuo to afford the crude product as a orange to brown oil. The product was purified via column chromatography (0-15% ethyl acetate/heptane), and the product containing fractions were combined and isolated to provide 14. HRMS: calculated for C₄₀H₅₅N₂O₆Si₂⁺: 715.3593 found: 715.3606.

### Example 8C

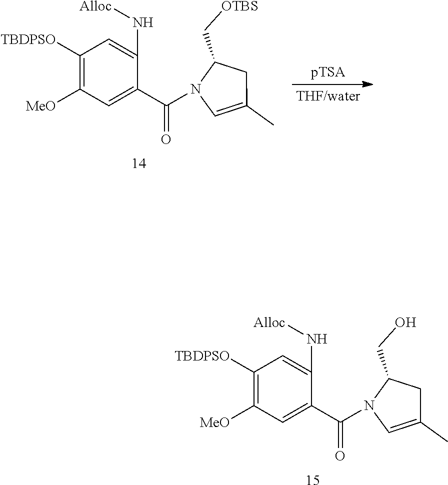

allyl(S)-(5-((tert-butyldiphenylsilyl)oxy)-2-(2-(hydroxymethyl)-4-methyl-2,3-dihydro-1H-pyrrole-1-carbonyl)-4-methoxyphenyl) carbamate (15): A solution of 14 (246.4 mg, 0.34 mmol, 1.0 equiv) in THF (6 vol) and and water (0.3 vol) was prepared. The solution was charged with p-toluenesulfonic acid hydrate (39.3 mg, 0.21 mmol, 0.6 equiv) and stirred at room temperature until the reaction was complete by HPLC. The reaction mixture was diluted with ethyl acetate (10 vol) and washed with water (4 vol) then brine (4 vol). The organic phase was collected, dried over sodium sulfate, and the filtered. The solution was concentrated in vacuo to afford the crude product as a orange to brown oil. The product was purified via column chromatography (0-15% ethyl acetate/heptane), and the product containing fractions were combined and isolated to provide 15. HRMS: calculated for C₃₄H₄₁N₂O₆Si₂⁺: 601.2728 found: 601.2735.

### Example 8D

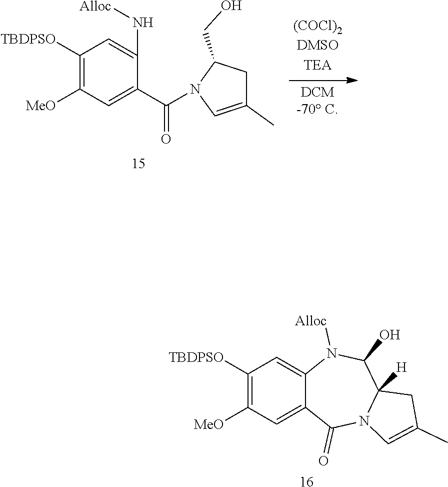

allyl(11S,11aS)-8-((tert-butyldiphenylsilyl)oxy)-11-hydroxy-7-methoxy-2-methyl-5-oxo-11,11a-dihydro-1H-benzo[e]pyrrolo[1,2-a][1,4]diazepine-10 (5H)-carboxylate (16): A solution of oxalyl chloride (9.3 UL, 0.11 mmol, 1.03 equiv) in DCM (9 vol) was prepared and cooled to −65° C. Dimethyl sulfoxide (18.3 μL, 0.26 mmol, 2.5 equiv) was added to the cooled solution. The reaction mixture was stirred for 30 min, and then a solution of 15 (62.1 mg, 0.10 mmol, 1.0 equiv) in DCM (8 V) was added dropwise. After 30 min, triethylamine (72.0 μL, 0.52 mmol, 5.0 equiv) was added dropwise. The reaction mixture was allowed to reach room temperature and stirred until complete by HPLC. The reaction was quenched with 5% citric acid (10 vol) and washed with saturated aqueous sodium bicarbonate (10 vol) and brine (10 vol). The organic phase was collected, dried over sodium sulfate, and the filtered. The solution was concentrated in vacuo to afford the crude product 17 which was used in the next step without purification (87% yield). HRMS: calculated for C₃₄H₃₉N₂O₆Si⁺: 599.2572 found: 599.2549.

### Example 8E

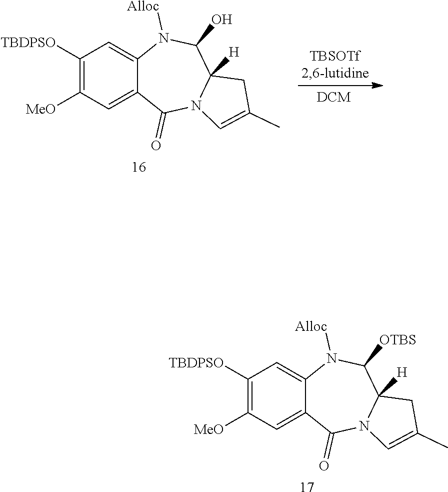

allyl(11S, 11aS)-11-((tert-butyldimethylsilyl)oxy)-8-((tert-butyldiphenylsilyl)oxy)-7-methoxy-2-methyl-5-oxo-11, 11a-dihydro-1H-benzo[e]pyrrolo[1,2-a][1,4]diazepine-10 (5H)-carboxylate (17): A solution of 16 (54.1 mg, 0.09 mmol, 1.0 equiv) in DCM (10 vol) was charged with 2,6-lutidine (71.8 μL, 0.62 mmol, 6.0 equiv) and cooled to 0° C. The chilled solution was charged with tert-butyldimethylsilyltriflate (119 μL, 0.52 mmol, 5.0 equiv). The reaction mixture was stirred at 0° C. for 30 min followed by 3 hr at room temperature. Once the reaction was complete by HPLC, it was quenched with saturated aqueous sodium bicarbonate (10 vol) and brine (10 vol). The organic phase was collected, dried over sodium sulfate, and the filtered. The solution was concentrated in vacuo to afford the crude product 17 which was used in the next step without purification. HRMS: calculated for C₄₀H₅₃N₂O₆Si₂⁺: 713.3437 found: 713.3429.

### Example 8F

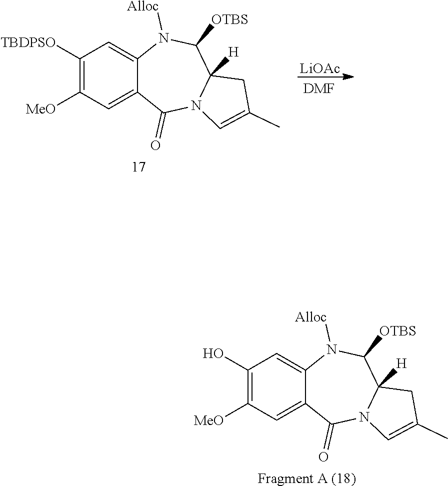

allyl(11S,11aS)-11-((tert-butyldimethylsilyl)oxy)-8-hydroxy-7-methoxy-2-methyl-5-oxo-11, 11a-dihydro-1H-benzo[e]pyrrolo[1,2-a][1,4]diazepine-10 (5H)-carboxylate (18): A solution of 17 (64.2 mg, 0.09 mmol, 1.0 equiv) in wet DMF (15 vol) was charged with lithium acetate (5.94 mg, 0.09 mmol, 1.0 equiv) and heated to 40° C. The reaction mixture was stirred at 40° C. until complete by HPLC. The reaction mixture was diluted with ethyl acetate (20 vol) and washed with water (10 vol) and saturated brine (10 vol). The organic phase was collected, dried over sodium sulfate, and the filtered. The solution was concentrated in vacuo to afford the crude product 18 which was consistent with the literature.2 HRMS: calculated for C₂₄H₃₅N₂O₆Si⁺: 475.2259 found: 475.2284.

### Example 8G

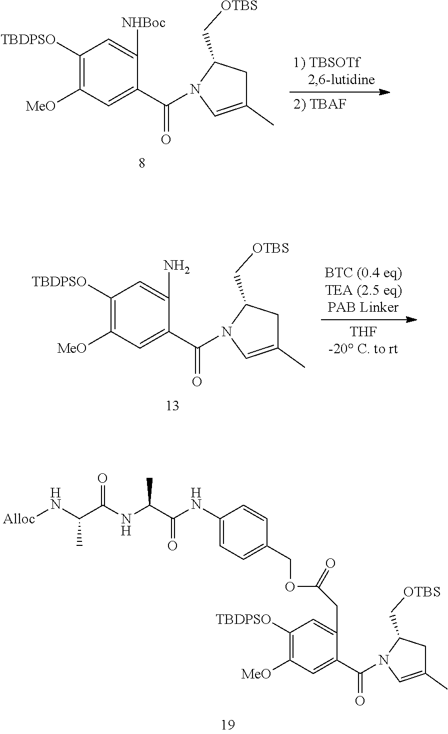

allyl((S)-1-(((S)-1-((4-((((2-((S)-2-(((tert-butyldimethylsilyl)oxy)methyl)-4-methyl-2,3-dihydro-1H-pyrrole-1-carbonyl)-5-((tert-butyldiphenylsilyl)oxy)-4-methoxyphenyl) carbamoyl)oxy)methyl)phenyl)amino)-1-oxopropan-2-yl)amino)-3-methyl-1-oxobutan-2-yl) carbamate (19) A solution of 8 (288.9 mg, 0.40 mmol, 1.0 equiv) in DCM (10 vol) was prepared. The solution was charged with 2,6-lutidine (0.91 mL, 7.9 mmol, 20 equiv) and TBSOTf (10.0 equiv). The solution is then heated to 40° C. and stirred for 15 h. When complete by HPLC, the mixture was quenched with saturated ammonium chloride (5 vol). The aqueous layer was back-extracted with DCM (5 vol), and the organic layers are combined, washed with saturated brine (5 vol), and dried over magnesium sulfate. The solution is concentrated in vacuo. The carbamate was taken up in THF (10 vol) and cooled to 0° C. The solution is charged with TBAF (0.16 mL, 1 M, 0.16 mmol, 0.4 equiv) and stirred at 0° C. for 30 min. The solution was then diluted with 10 vol of ethyl acetate and quenched with 10 vol of water. The layers are separated, and the aqueous layer is extracted with ethyl acetate (10 vol). The organic layers are combined, dried over magnesium sulfate, and concentrated in vacuo to provide 14. The material was used immediately to avoid decomposition. 13 was charged with 10 vol of DCM and cooled to −20° C. Triphosgene (46.9 mg, 0.16 mmol, 0.40 equiv) was slowly added to the reaction mixture. The solution was then charged with triethylamine (121 μL, 0.87 mmol, 2.2 equiv) and stirred for 30 min at −20° C. Meanwhile, a mixture of allyl((S)-1-(((S)-1-((4-(hydroxymethyl)phenyl)amino)-1-oxopropan-2-yl)amino)-3-methyl-1-oxobutan-2-yl) carbamate (156.6 mg, 0.41 mmol, 1.05 equiv) in triethylamine (83 μL, 0.60 mmol, 1.5 equiv) in DCM (10 vol) was prepared. This solution was rapidly charged to the reaction at −20° C. The reaction was allowed to stir for 15 h. When the reaction was complete by HPLC, the reaction mixture was washed with water (4 vol). The organic phase was collected, dried over sodium sulfate, and concentrated in vacuo. The resulting mixture as purified via flash chromatography to provide the product as a white solid (321.2 mg, 79% yield over 3 steps). HRMS: calculated for C₅₆H₇₆N₅O₁₀Si₂⁺: 1034.5125 found: 1034.5094.

### Example 8H

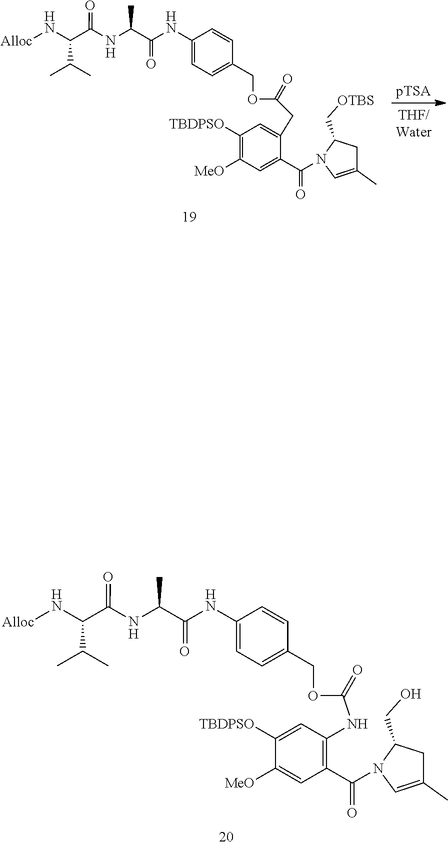

allyl((S)-1-(((S)-1-((4-((((5-((tert-butyldiphenylsilyl)oxy)-2-((S)-2-(hydroxymethyl)-4-methyl-2,3-dihydro-1H-pyrrole-1-carbonyl)-4-methoxyphenyl) carbamoyl)oxy)methyl)phenyl)amino)-1-oxopropan-2-yl)amino)-3-methyl-1-oxobutan-2-yl) carbamate (20) A solution of 19 (321.2 mg, 0.31 mmol, 1.0 equiv) in THF (6 vol) and water (0.3 vol) was prepared. The solution was charged with para-toluenesulfonic acid hydrate (35.4 mg, 0.18 mmol, 0.6 equiv) for 2 hrs. When the reaction was complete by HPLC, the solution was diluted with ethyl acetate (10 vol) and washed with water (5 vol), saturated sodium bicarbonate (5 vol), and saturated brine (5 vol). The organic phase was collected, dried over sodium sulfate, and concentrated in vacuo. The product was purified via column chromatography (heptane/ethyl acetate 40/60 to 0/100) to afford the product as a slightly yellow solid (83% yield). HRMS: calculated for C₅₀H₆₂N₅O₁₀Si⁺: 920.4260 found: 920.4264.

### Example 81

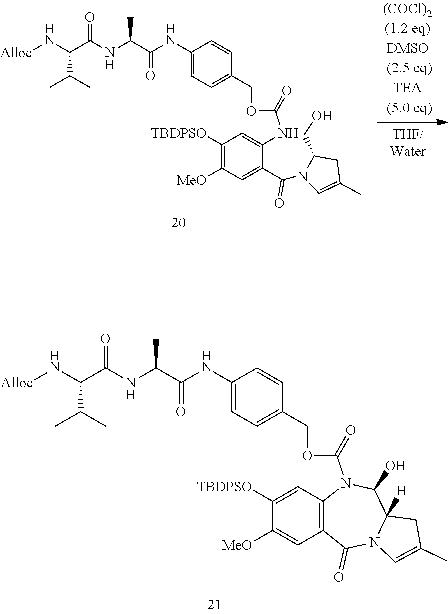

4-((S)-2-((S)-2-(((allyloxy) carbonyl)amino)-3-methylbutanamido) propanamido)benzyl(11S, 11aS)-8-((tert-butyldiphenylsilyl)oxy)-11-hydroxy-7-methoxy-2-methyl-5-oxo-11,11a-dihydro-1H-benzo[e]pyrrolo[1,2-a][1,4]diazepine-10 (5H)-carboxylate (21) A solution of oxalyl chloride (2.03 M, 152 μL, 0.31 mmol, 1.2 equiv) in DCM was prepared and cooled to −65° C. DMSO (46 μL, 0.64 mmol, 2.5 equiv) was added dropwise. The resulting solution was stirred for 15 min, at which point a solution of 20 (236.2 mg, 0.26 mmol, 1.0 equiv) in DCM (5 vol) was added dropwise. The reaction was stirred for 15 min at −65° C., and then triethylamine (179 μL, 1.28 mmol, 5.0 equiv) was added dropwise. The reaction was stirred for 1 hr at −65° C. Once the reaction was complete by HPLC, the reaction mixture was warmed to 0° C. and washed with 0.2 N HCl (10 vol), saturated sodium bicarbonate (10 vol), and brine (10 vol). The organic phase was collected and concentrated in vacuo. The residue was then charged with DCM (10 vol), and the resulting solution was again concentrated in vacuo. This procedure was repeated twice to afford the product 21 in 86% yield. The crude product was immediately used in the next step. HRMS: calculated for C₅₀H₆₀N₅O₁₀Si⁺: 918.4104 found: 918.4091.

### Example 8J

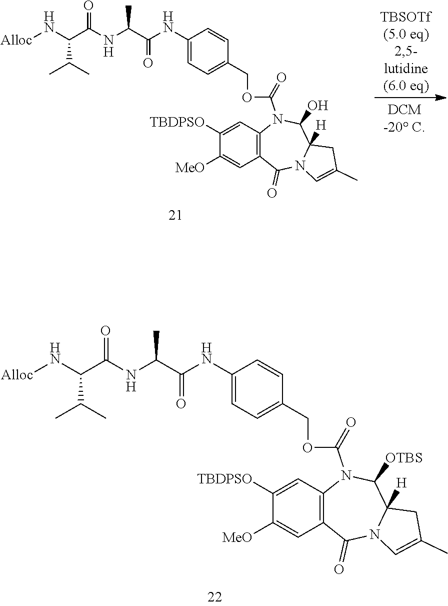

4-(S)-2-((S)-2-(((allyloxy) carbonyl)amino)-3-methylbutanamido) propanamido)benzyl(11S)-11-((tert-butyldimethylsilyl)oxy)-8-((tert-butyldiphenylsilyl)oxy)-7-methoxy-2-methyl-5-oxo-11,11a-dihydro-1H-benzo[e]pyrrolo[1,2-a][1,4]diazepine-10 (5H)-carboxylate (22) A solution of 21 (203 mg, 0.22 mmol, 1.0 equiv) in DCM (20 vol) was cooled to −15° C. and charged with 2,6-lutidine (178 μL, 1.54 mmol, 6.0 equiv). The reaction mixture was cooled to −40° C. and tert-butyldimentylsilyltriflate (295 μL, 1.28 mmol, 5.0 equiv) was added dropwise. The reaction was stirred for 30 min at oxalyl chloride (2.03 M, 152 μL, 0.31 mmol, 1.2 equiv) in DCM was prepared and cooled to −65° C. DMSO (46 μL, 0.64 mmol, 2.5 equiv) was added dropwise. The resulting solution was stirred for 15 min at −40° C. followed by 20 hrs at −20° C. When the reaction was complete by HPLC, it was quenched with 10% citric acid (10 vol) and washed with saturated sodium bicarbonate (10 vol) and brine (10 vol). The organic phase was collected and concentrated in vacuo. The residue was then charged with DCM (10 vol), and the resulting solution was again concentrated in vacuo. This procedure was repeated twice to afford the product 22. The crude product was immediately used in the next step. HRMS: calculated for C₅₆H₇₄N₅O₁₀Si₂⁺: 1032.4969 found: 1032.4922.

### Example 8K

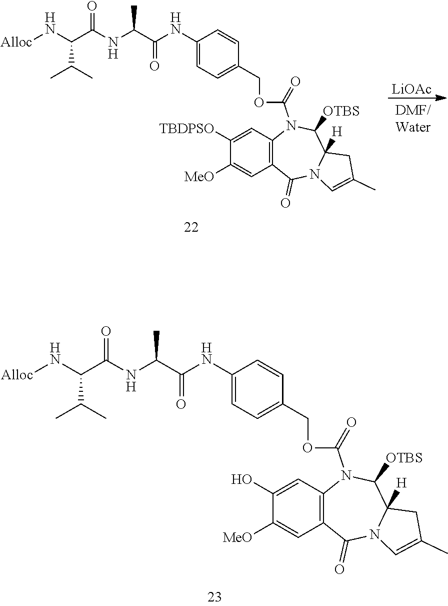

4-((S)-2-((S)-2-(((allyloxy) carbonyl)amino)-3-methylbutanamido) propanamido)benzyl(11S)-11-((tert-butyldimethylsilyl)oxy)-8-hydroxy-7-methoxy-2-methyl-5-oxo-11,11a-dihydro-1H-benzo[e]pyrrolo[1,2-a][1,4]diazepine-10 (5H)-carboxylate (23) A solution of crude 22 (265 mg, 0.26 mmol, 1.0 equiv) in DMF (10 vol) and water (0.3 equiv) was prepared. The solution was charged with lithium acetate (16.9 mg, 0.26 mmol, 1.0 equiv). The mixture was heated to 40° C. and stirred for 8 hrs. Once the reaction was complete by HPLC, the mixture was allowed to cool to room temperature and diluted with Me-THF (10 vol). The mixture was washed with 10% citric acid (10 vol), 5% sodium bicarbonate (10 vol), saturated brine (10 vol), and water (10 vol). The organic phase was collected and concentrated in vacuo. The residue was then charged with 2-MeTHF (10 vol), and the resulting solution was again concentrated in vacuo. This procedure was repeated twice to afford the crude product. The crude product was purified via column chromatography (heptane/ethyl acetate 40/60 to 0/100) to afford the product 23 (80.2 mg, 40% yield over two steps). The product matched reported NMR data^{1b, 2} HRMS: calculated for C₄₀H₅₅N₅O₁₀Si⁺: 794.3791 found: 794.3778.

### Example 8L

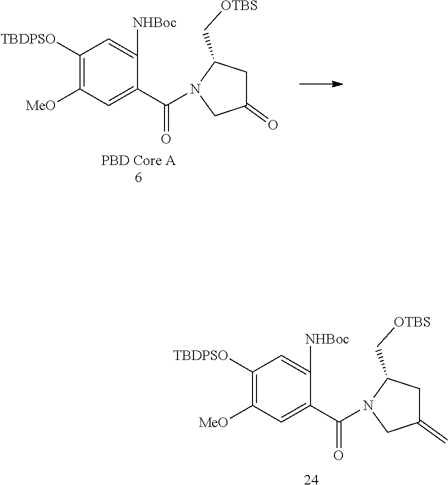

tert-butyl(S)-(2-(2-(((tert-butyldimethylsilyl)oxy)methyl)-4-methylenepyrrolidine-1-carbonyl)-5-((tert-butyldiphenylsilyl)oxy)-4-methoxyphenyl) carbamate (24) A solution of methyltriphenyl phosphonium bromide (2.92 g, 8.17 mmol, 2.0 equiv) in THF (38.5 vol) was prepared and cooled to 0° C. The solution was charged with potassium tert-butoxide (1.0 M, 8.18 mL, 8.18 mmol, 2.0 equiv) dropwise. The reaction mixture was stirred at 0° C. for 15 min, then room temperature for 45 min. The reaction mixture was then cooled to 0° C. and charged with PBD Core A (3.0 g, 4.09 mmol, 1.0 equiv) in THF (1.5 vol) dropwise. After the addition of PBD Core A was complete, the reaction mixture was allowed to warm to room temperature and stirred until the reaction was complete by HPLC. The reaction was quenched with water (15 vol). The aqueous layer was collected and washed twice with MTBE (15 vol). The organic phases were combined and washed with water (15 vol) and brine (15 vol), dried over sodium sulfate, then concentrated in vacuo. The product was purified via column chromatography (heptane/ethyl acetate 85/15). The product containing fractions were isolated to afford the product 24 (2.00 g, 2.7 mmol) in 64% yield. HRMS: calculated for C₄₁H₅₉N₂O₆Si₂⁺: 731.3906 found: 731.3786.

### Example 8M

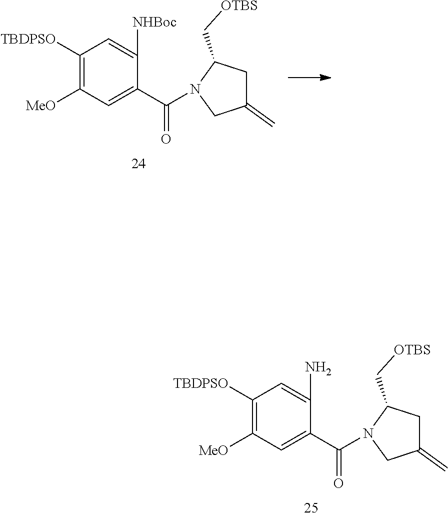

(S)-(2-amino-4-((tert-butyldiphenylsilyl)oxy)-5-methoxyphenyl) (2-(((tert-butyldimethylsilyl)oxy)-methyl)-4-methylenepyrrolidin-1-yl) methanone (25): A solution of 24 (300.0 mg, 0.40 mmol, 1.0 equiv) in DCM (5 vol) was prepared. The solution was charged with 2,6-lutidine (0.19 mL, 1.6 mmol, 20.0 equiv) and cooled to 0° C. The reaction mixture was then charged with tert-butyldimentylsilyltriflate (0.18 mL, 0.8 mmol, 2.0 equiv). The solution was stirred at 0° C. for 30 min, then room temperature for 24 h. The reaction was cooled to 0° C. and charged with 10% aqueous citric acid solution. The resulting mixture was stirred at room temperature for 96 hrs. The reaction mixture was diluted with DCM (3 vol). The organic layer was collected and washed with saturated brine (5 vol). The crude product was isolated (90% yield) and stored at −20° C. HRMS: calculated for C₃₆H₅₁N₂O₄Si₂⁺: 631.3382 found: 631.3354.

### Example 8N

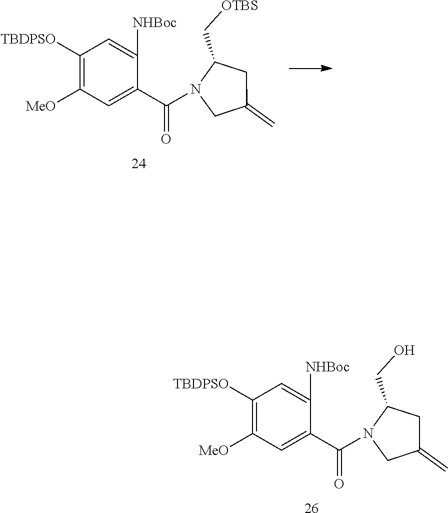

tert-butyl(S)-(5-((tert-butyldiphenylsilyl)oxy)-2-(2-(hydroxymethyl)-4-methylenepyrrolidine-1-carbonyl)-4-methoxyphenyl) carbamate (26): A solution of 24 (1.90 g, 2.6 mmol, 1.0 equiv) in ethyl acetate (15 vol) was cooled to 0° C. The solution was charged with hydrochloric acid (37% reagent, 0.03 mL, 0.8 mmol, 0.3 equiv). Reaction was allowed to slowly warm to room temperature and stirred for 2 hrs. The reaction was complete by HPLC. Half the material was set aside for stability studies. The remaining half was isolated and subjected to the Swern conditions (see the preparation of 27). HRMS: calculated for C₃₅H₄₅N₂O₆Si⁺: 617.3041 found: 617.3065.

### Example 80

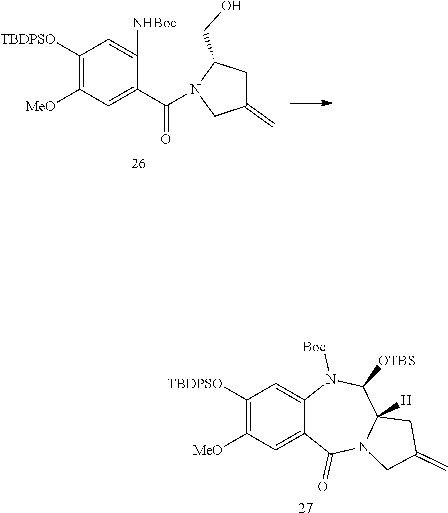

tert-butyl(11S, 11aS)-8-((tert-butyldiphenylsilyl)oxy)-11-hydroxy-7-methoxy-2-methylene-5-oxo-2,3,11, 11a-tetrahydro-1H-benzo[e]pyrrolo[1,2-a][1,4]diazepine-10 (5H)-carboxylate (27): A solution of 26 (0.65 g, 1.1 mmol, 1.0 equiv) in THF (10 vol) is charged with DMSO (0.19 mL, 2.6 mmol, 2.5 equiv) and cooled to −78° C. The reaction mixture was charged with trifluoroacetic anhydride (0.16 mL, 1.2 mmol, 1.1 equiv) was added dropwise then stirred at −78° C. for 30 min. Triethylamine (0.73 mL, 5.3 mmol, 5.0 equiv) was then slowly added to the reaction mixture. The reaction was then quenched with 5% aqueous citric acid solution and stirred for 15 min at 5° C. The organic phase was collected and washed with saturated sodium bicarbonate and water, then dried over magnesium sulfate. The intermediate was isolated and charged with DCM (10 vol). The solution was cooled to −78° C. and charged with 2,6-lutidine (0.31 mL, 2.6 mmol, 2.5 equiv) followed by tert-butyldimethylsilyl trifluoromethanesulfonate (0.36 mL, 1.6 mmol, 1.5 equiv). The reaction was allowed to slowly warm to room temperature over 30 min. The reaction was allowed to stir at room temperature for 15 hr. When the reaction was complete by HPLC, the reaction mixture was diluted with DCM (45 vol) and quenched with saturated sodium bicarbonate. The organic layer was collected, washed with brine, and dried over sodium sulfate. The crude product was isolated in vacuo and purified via column chromatography (heptane/ethyl acetate 75/25). The product-containing fractions were combined to afford 27 in 54% yield over 3 steps. HRMS: calculated for C₄₁H₅₇N₂O₆Si₂⁺: 729.3750 found: 729.3790.

### Example 8P

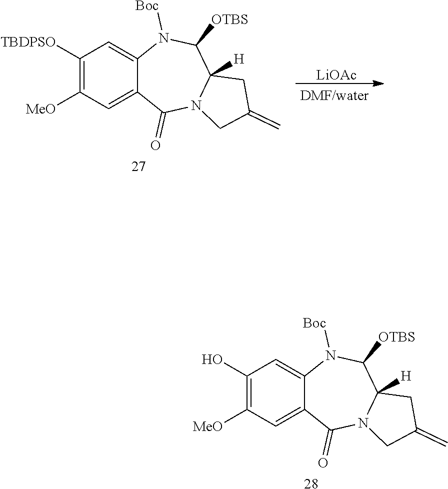

tert-butyl(11S, 11aS)-11-((tert-butyldimethylsilyl)oxy)-8-hydroxy-7-methoxy-2-methylene-5-oxo-2,3, 11, 11a-tetrahydro-1H-benzo[e]pyrrolo[1,2-a][1,4]diazepine-10 (5H)-carboxylate (28) A solution of crude 27 (205 mg, 0.28 mmol, 1.0 equiv) in DMF (10 vol) and water (0.3 equiv) was prepared. The solution was charged with lithium acetate (18 mg, 0.28 mmol, 1.0 equiv). The mixture was stirred for 15 h at room temperature. Once the reaction was complete by HPLC, the reaction mixture was diluted with ethyl acetate (25 vol) and washed twice with water (20 vol) and twice with brine (20 vol). The organic phase was collected, dried over sodium sulfate, and concentrated in vacuo. The crude product was purified via column chromatography (heptane/ethyl acetate 30/70) to afford the product 28 (80.2 mg, 40% yield over two steps). The product matched reported NMR data^{1b, 2} HRMS: calculated for C₂₅H₃₉N₂O₆Si⁺: 491.2572 found: 491.2520.

The examples herein are for illustrative purposes only and are not meant to limit the scope of the invention as defined by the claims.

### REFERENCES

(a) Gregson, S. J.; Howard, P. W.; Gullick, D. R.; Hamaguchi, A.; Corcoran, K. E.; Brooks, N. A.; Hartley, J. A.; Jenkins, T. C.; Patel, S.; Guille, M. J.; Thurston, D. E. J. Med. Chem. 2004, 57 (5), 1161-1174; (b) Tiberghien, A. C.; von Bulow, C.; Barry, C.; Ge, H.; Noti, C.; Leiris, F. C.; McCormick, M.; Howard, P. W.; Parker, J. S. Org. Process Res. Dev. 2018, 22 (9), 1241-1256.
(a) Tiberghien, A. C.; Levy, J.-N.; Masterson, L. A.; Patel, N. V.; Adams, L. R.; Corbett, S.; Williams, D. G.; Hartley, J. A.; Howard, P. W. ACS Med. Chem. Lett. 2016, 7 (11), 983-987; (b) Tiberghien, A. C.; Howard, P. W.; Goundry, W. R. F.; McCormick, M.; Parkers, J. S. J. Org. Chem. 2019, 84 (8), 4830-4836; (c) Lai, W.; Zhao, S.; Lai, Q.; Zhou, W.; Wu, M.; Jiang, X.; Wang, X.; Peng, Y.; Wei, X.; Ouyang, L.; Gou, L.; Chen, H.; Wang, Y.; Yang, J. J. Med. Chem. 2022, 65 (17), 11679-11702.

1. A compound of formula I 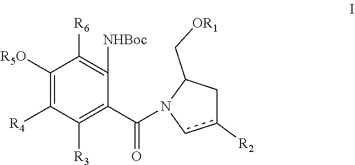
wherein
the broken line represents a single or double bond,
R₁ is a protecting group selected from tert-butyldimethylsilyl ether (TBS) and acetate (Ac),
R₂ is selected from the group consisting of H, ═O, —OTf, C₁-C₅ alkyl, C₃-C₆ cycloalkyl, C₁-C₅ alkenyl, C₅-C₁₀ aryl, C₅-C₁₀ heteroaryl, and a linker, wherein R₂ may be optionally substituted with one or more substituents selected from the group consisting of halo, nitro, cyano, ether, carboxy, ester, C₁-C₇ alkyl, C₁-C₇ alkenyl, C₃-C₇ heterocyclyl, bis-oxy-C₁-C₃ alkylene and a linker,
R₃ is selected from the group consisting of H, —OH, —OR, —SH, —NH₂, —NO₂, halo, wherein R is selected from the group consisting of C₁-C₄ alkyl, C₃-C₁₀ heterocyclyl and C₅-C₁₀ aryl, and R may optionally have a substituent selected from the group consisting of C₁-C₄ alkyl and phenyl,
R₄ is selected from the group consisting of H, —R, —OH, —OR, —SH, —SR, —NH₂, —NHR, —NHRR′, nitro and halo, wherein R and R′ are independently selected from the group consisting of C₁-C₄ alkyl, C₃-C₁₀ heterocyclyl and C₅-C₁₀ aryl, and R and R′ may optionally have a substituent selected from the group consisting of C₁-C₄ alkyl and phenyl,
R₅ is a protecting group selected from tert-butyldiphenylsilyl ether (TBDPS) and triisopropylsilyl ether (TIPS), and
R₆ is selected from the group consisting of —OH, —OR, —SH, —NH₂, —NO₂, halo, wherein R is selected from the group consisting of C₁-C₄ alkyl, C₃-C₁₀ heterocyclyl and C₅-C₁₀ aryl, and R may optionally have a substituent selected from the group consisting of C₁-C₄ alkyl, phenyl.

2. The compound of claim 1 wherein R₃ and R₆ are each H.

3. The compound of claim 2 wherein R₂ is selected from the group consisting of H, ═O, —OTf, —OH, methyl, cyclopropyl, ═CH₂, ethylenyl, propenyl, phenyl, 4-fluorophenyl, indole and a linker, and R₂ may be optionally substituted with one or more substituents selected from the group consisting of halo, nitro, cyano, ether, carboxy, ester, C₁-C₇ alkyl, C₁-C₇ alkenyl, C₃-C₇ heterocyclyl, bis-oxy-C₁-C₃ alkylene and a linker, and
R₄ is selected from the group consisting of H, —OCH₃, —OCH₂Ph, —N(CH₃)₂, morpholino, piperidinyl, and —N—Me-piperazinyl.

4. The compound of claim 3 wherein R₂ is ═O.

5. The compound of claim 3 wherein R₂ is —OTf.

6. The compound of claim 1 wherein R₄ is OCH₃.

7. The compound of claim 4 selected from

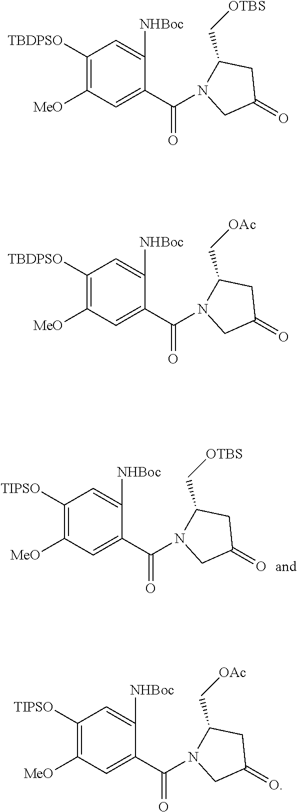

8. The compound of claim 5 selected from

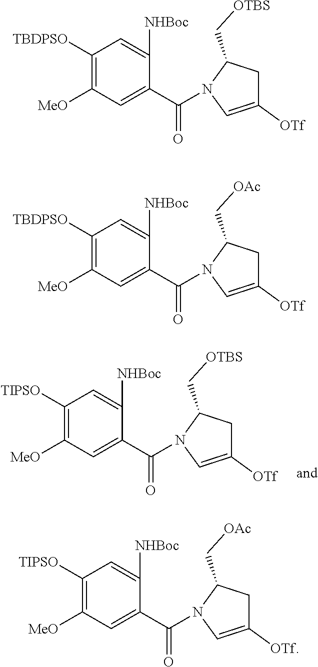

9. The compound of claim 1 selected from

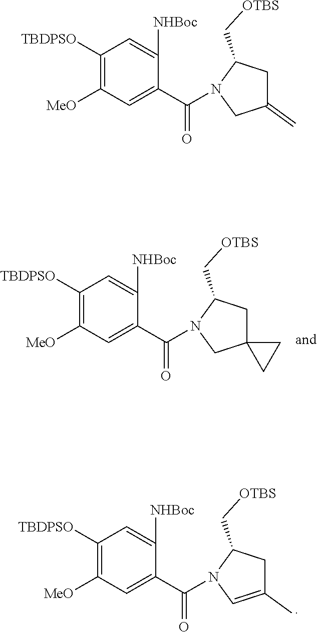

10. A compound selected from

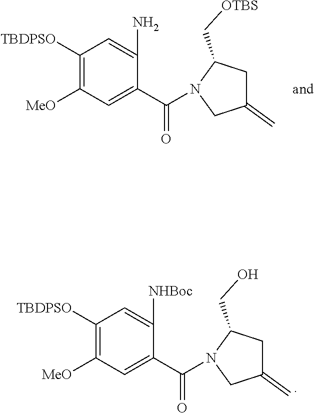

11. A compound of formula II: 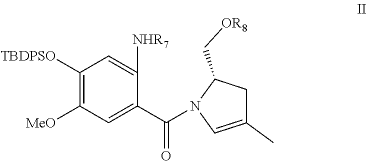
wherein R₇ is selected from the group consisting of H, —C(O)O-TBS, Alloc, and linkers, and R₅ is selected from H and TBS.

12. The compound of claim 11 selected from

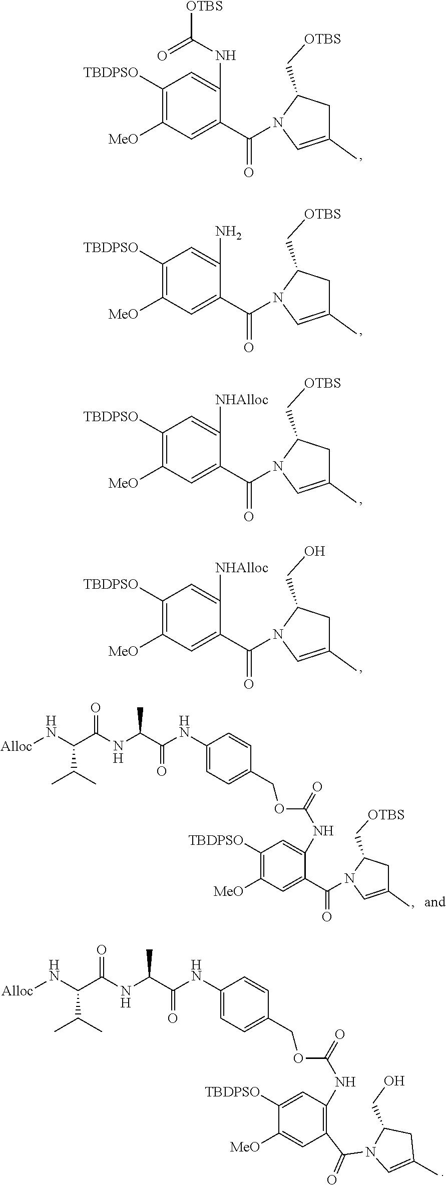

13. A compound of formula III: 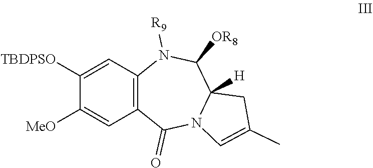
wherein R₉ is selected from Alloc and a linker, and R₅ is selected from H and TBS.

14. The compound of claim 13 selected from:

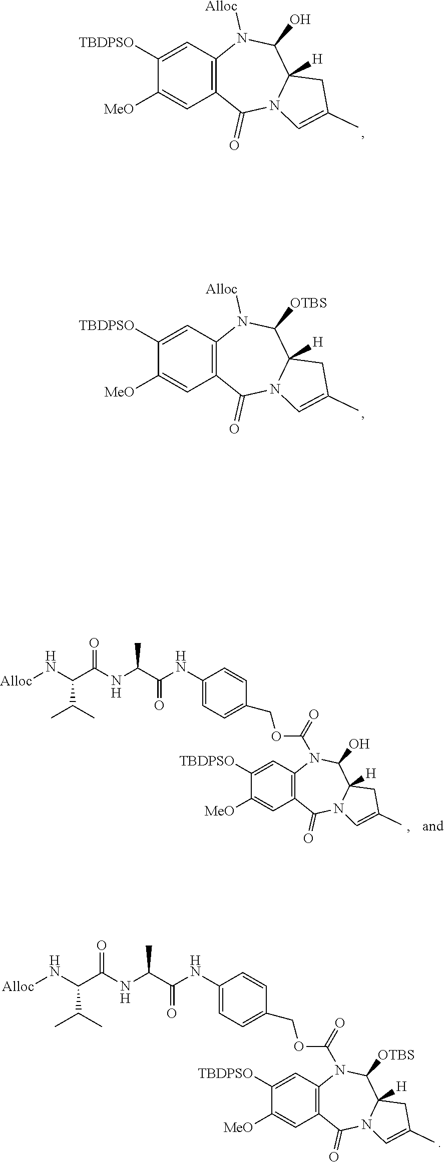

15. A method for making an advanced pyrrolobenzodiazepine (PBD) intermediate comprising the steps of
providing a compound of claim 4,
performing a C—C bond forming reaction at the ketone, and
optionally, deprotecting the amine.

16. A method for making an advanced PBD intermediate comprising the steps of
providing a compound of claim 4,
converting the ketone to a triflate,
performing a C—C bond forming reaction on the triflate, and
optionally, deprotecting the amine.

17. The method of claim 15 wherein the C—C bond forming reaction is Suzuki coupling.

18. A method for making an advanced PBD intermediate comprising the steps of
providing a compound of claim 5,
performing a C—C bond forming reaction on the triflate, and optionally, deprotecting the amine.

19. The method of claim 18 wherein the C—C bond forming reaction is Suzuki coupling.

20. The method of claim 15, wherein the amine is deprotected, further comprising the step of coupling a linker to the deprotected amine.
